(19) United States
(12) Patent Application Publication
(10) Pub. No.: US 2015/0087902 A1
(43) Pub. Date: Mar. 26, 2015

(54) Phase Contrast Microscopy With Oblique Back-Illumination

(71) Applicant: Trustees of Boston University, Boston, MA (US)

(72) Inventors: Jerome Charles Mertz, Newton, MA (US); Timothy Nehiley Ford, Allston, MA (US); Kengyeh Ken Chu, Jamaica Plain, MA (US)

(21) Appl. No.: 14/388,847

(22) PCT Filed: Mar. 15, 2013
(86) PCT No.: PCT/US2013/032517

§ 371 Date: Sep. 29, 2014

Related U.S. Application Data
(60) Provisional application No. 61/617,707, filed on Mar. 30, 2012.

(51) Int. Cl. G06T 5/00; G02B 21/00; A61B 1/00; G01N 21/25; A61B 1/04; G02B 21/06; G01N 21/27

(52) U.S. Cl. CPC G06T 5/009; G02B 21/06; G02B 21/0012; G01N 21/27; G01N 21/255; A61B 1/04; A61B 1/00009; A61B 1/00172; G01N 2201/062; G01N 2201/0866; G06T 2207/10068; G06T 2207/20208; G06T 2207/20221
USPC 600/109; 348/79; 348/68

(57) ABSTRACT

A method of creating a phase contrast image is provided. In some embodiments the method comprises illuminating the target region of a sample with a first light source to provide a first oblique back illumination of the target region of the sample, and detecting a first phase contrast image from light originating from the first light source and back illuminating the target region of the sample. In some embodiments the method further comprises illuminating the sample with a second light source to provide a second oblique back illumination of the target region of the sample, and detecting a second phase contrast image from light originating from the second light source and back illuminating the target region of the sample. In some embodiments a difference image of the target region of the sample is created by subtracting the second phase contrast image of the target region of the sample from the first phase contrast image of the target region of the sample. Apparatus for carrying out the methods are also provided. The methods and apparatus find use, for example, in endoscopy.

### CROSS-REFERENCE TO RELATED APPLICATIONS

This application claims priority to U.S. Provisional Application No. 61/617,707, filed Mar. 30, 2012, which is hereby incorporated herein by reference.

### GOVERNMENT FUNDING

This invention was made with Government Support under Contract No. EB010059 awarded by the National Institutes of Health. The Government has certain rights in the invention.

### INTRODUCTION

The standard technique to assess tissue pathology in clinical applications is to perform a biopsy [1]. In general, assessment is made based on purely morphological considerations. The technique often involves use of a device to observe tissue with high resolution. As successful and prevalent as this biopsy procedure has become, it faces certain drawbacks. For example, the process is laborious and time consuming, requiring hours or days to provide results. For certain applications it would be useful to have an alternative procedure that, in some embodiments, requires less time and/or work. For another example, tissue biopsies only provide a sparse sampling that may not be fully representative of the region of interest. For certain applications it would be useful to have an alternative procedure that, in some embodiments, enables more comprehensive sampling of the region of interest. For another example, tissue biopsies pose a risk of infection and/or other complications to the patient and can cause discomfort. For certain applications it would be useful to have an alternative procedure that, in some embodiments, causes less discomfort and/or has a reduced risk of infection and/or other complications to the patient.

The concept of an “optical biopsy” has long been sought by the biomedical imaging community [2], [3]. Nonetheless, efforts to develop optical biopsy techniques and equipment have had a limited success. Many strategies for optical biopsies have been proposed. In general, these can be separated into two broad categories, those based on imaging and those based on spectroscopy [3].

Phase contrast imaging is one of the most prevalent applications of wide field microscopy, and there exists an abundance of literature describing different wide field phase contrast techniques. The most common of these, found in virtually every cellular biology lab, are Zernike phase contrast [4] or Normarski differential interference contrast (DIC)[5], [6]. The latter is also widely used in neurophysiology labs, since it is highly effective at revealing neurons in brain tissue slices. Other wide field phase contrast techniques include Schlieren microscopy [7], Hoffman contrast [8], or other variants of oblique field microscopies such as field contrast [9], [10]. None of these techniques is particularly quantitative in the sense that the measured signal cannot be easily converted into a measured phase. Nevertheless, the signals are phase dependent, and thus reveal variations in optical path length. Only recently (relatively speaking) has there been a trend toward the development of phase contrast techniques that are genuinely quantitative ([11-16]). The application of these techniques is limited, however, because each one works only in the transmission direction. This feature limits the use of these techniques to use with a transmission light source. The techniques are therefore applied to thin samples, such as cell monolayers or thin tissue slices.

Other techniques also work in the reflection direction, such as reflection confocal microscopy [17]. In this technique, signal arises from local reflectivities in the sample, which, in turn arise from refractive index variations. A difficulty with reflection confocal is that scattering in most biological tissues is dominantly in the forward direction. Only sharp interfaces (i.e. refractive index variations with high enough axial spatial frequencies) produce scattering in the backward direction, meaning that signal is weak. Moreover, the signal can easily be overwhelmed by multiply scattered light containing no image information. Both of these problems are solved by optical coherence tomography (OCT), which provides noiseless amplification of the directly back-scattered signal while rejecting multiply scattered background [18]. Nevertheless, the fact remains that OCT, like reflectance confocal, reveals mostly sharp axial interfaces in samples.

In most of their incarnations, reflectance confocal and OCT are based on scanning geometries, and thus require scanning mechanisms, somewhat complicating their operation in endoscopy applications. Certain designs have been proposed to overcome this feature [19-21].

In addition to OCT, other techniques have been developed to provide high resolution imaging in thick tissue. Examples are photo-acoustic microscopy (PAM) [23] and nonlinear microscopy [24], e.g. based on two-photon excited fluorescence (TPEF) or second harmonic generation (SHG). PAM reveals absorbing structures, TPEF reveals fluorophores, and SHG reveals non-centrosymmetric structures. None provides phase contrast in the usual sense of the term. Moreover, PAM, TPEF and SHG are all scanning techniques.

Another class of thick tissue imaging techniques uses the detection of multiply scattered light. Examples are diffuse optical tomography (DOT) [30], and a beam scanning variant, laminar optical tomography (LOT) [31]. Image reconstruction with these techniques is based on mathematical models, and the extraction of data usually requires intensive numerical processing. These techniques can provide very deep tissue penetration, but it occurs at the expense of resolution. They reveal tissue properties such as absorption and/or scattering coefficients. They provide neither high resolution nor phase contrast. Nor have they been applied in endoscopy configurations.

Another technique is Orthogonal Polarization Spectral (OPS) imaging [32], [33], now commercialized as Cytoscan™ microscopy. This strategy is similar to OBM in that it generates backlighting from multiply scattered light (launched on-axis and distinguished by the fact that it is depolarized). This technique uses shadow-casting to reveal absorption contrast only. This technique cannot reveal phase contrast. Moreover, it provides only low resolution images with a rigid, handheld probe, and it cannot be combined with standard endoscopes.

Accordingly, there is a need for new imaging methods and apparatus that have useful properties. This disclosure meets that need by describing new imaging methods and apparatus, among other things.

### SUMMARY

This disclosure describes a new phase contrast technique, sometimes referred to herein as oblique back-illumination microscopy (OBM). OBM works in a reflected light geometry (sometimes called epi-detection geometry), and is thus amenable to in-vivo endomicroscopy applications, among many others. OBM requires no labeling and provides high resolution DIC-like images of sub-surface sample morphology. As will become apparent from this disclosure, the methods and apparatus disclosed herein apply the new OBM technology in ways that offer useful improvements in various ways to other technologies currently available.

In a first aspect this disclosure provides methods of creating a phase contrast image, comprising: illuminating the target region of a sample with a first light source to provide a first oblique back illumination of the target region of the sample, and detecting a first phase contrast image from light originating from the first light source and back illuminating the target region of the sample. In some embodiments light from the first light source is the only light illuminating the sample when the first phase contrast image is detected from light originating from the first light source and back illuminating the target region of the sample.

In some embodiments the methods further comprise illuminating the sample with a second light source to provide a second oblique back illumination of the target region of the sample, and detecting a second phase contrast image from light originating from the second light source and back illuminating the target region of the sample. In some embodiments the methods further comprise creating a difference image of the target region of the sample by subtracting the second phase contrast image of the target region of the sample from the first phase contrast image of the target region of the sample. In some embodiments the methods further comprise creating an absorption contrast image of the target region of the sample by adding the first phase contrast image of the target region of the sample to the second phase contrast image of the target region of the sample.

In some embodiments the axis of illumination of the sample with the first light source and the axis of detection of light originating from the first light source and back illuminating the target region are different. In some embodiments the axis of illumination of the sample with the second light source and the axis of detection of light originating from the second light source and back illuminating the target region are different. In some embodiments the axis of detection of light originating from the first light source and back illuminating the target region and the axis of detection of light originating from the second light source and back illuminating the target region are different. In some embodiments the axis of detection of light originating from the first light source and back illuminating the target region and the axis of detection of light originating from the second light source and back illuminating the target region are the same. In some embodiments the wavelength of the light from the first light source and the wavelength of light from the second light source are different. In some embodiments the wavelength of the light from the first and second light sources is from 0.2 to 300 μm. In some embodiments the light source(s) is selected from a light-emitting diode (LED), a laser, a supercontinuum light source, or a superluminescent diode (SLED). In some embodiments the detecting is by a photo detector array. In some embodiments the photo detector array is a charge coupled device (CCD) or a CMOS (complementary metal oxide semiconductor) camera sensor.

In some embodiments the methods comprise using an optical conduit to communicate light in at least one direction selected from toward the sample and away from the sample. In some embodiments the optical conduit to communicate light in at least one direction selected from toward the sample and away from the sample is selected from a fiber, an arrangement of fibers, a fiber bundle, a rigid lens, an arrangement of rigid lenses, a gradient index (GRIN) lens, or an arrangement of GRIN lenses. In some embodiments the same optical conduit communicates light toward the sample and away from the sample. In some embodiments different components of the same optical conduit communicates light toward the sample and away from the sample.

In some embodiments the axis of illumination of the sample with the first light source and the axis of detection of light originating from the first light source are displaced by from about 0.2 mm to about 10 mm. In some embodiments the axis of illumination of the sample with the second light source and the axis of detection of light originating from the second light source are displaced by from about 0.2 mm to about 10 mm.

In some embodiments the object plane of the target region is from the surface to about 300 μm below the surface of the sample. In some embodiments the lateral resolution of the image is from about 0.1 μm to about 10 μm.

In some embodiments the methods comprise detecting the first and second images during first and second non-overlapping time intervals. In some embodiments the methods comprise detecting the first and second images during first and second overlapping time intervals.

In some embodiments the first and second light sources illuminate the sample with light of different distinguishable wavelengths. In some embodiments the images of different distinguishable wavelengths are separated by a wavelength separator and directed onto separate camera sensors. In some embodiments the images of different distinguishable wavelengths are separated by a wavelength separator and directed onto different portions of a same camera sensor.

In some embodiments the first and second light sources illuminate the sample with orthogonally polarized light. In some embodiments the images of orthogonal polarization are separated by a polarization separator and directed onto separate camera sensors. In some embodiments the images of orthogonal polarization are separated by a polarization separator and directed onto different portions of a same camera sensor.

In some embodiments the difference image is axially resolved.

In some embodiments the methods comprise obtaining a series of two or more images and combining the images to provide a composite image larger than the field of view a single image. In some embodiments the methods comprise creating a phase contrast image of gastrointestinal tissue and examining the tissue to assess at least one of the presence and the absence of indicators of a disease. In some embodiments the gastrointestinal tissue is colonic mucosa disease is at least one of hyperplasia and adenomatous changes. In some embodiments the methods comprise creating a phase contrast image of lung tissue and examining the tissue to assess at least one of the presence and the absence of at least one indicator of a disease. In some embodiments the methods comprise creating a phase contrast image of liver tissue and examining the tissue to assess at least one of the presence and the absence of at least one indicator of a disease. In some embodiments the methods comprise creating a phase contrast image of bladder tissue and examining the tissue to assess at least one of the presence and the absence of at least one indicator of a disease. In some embodiments the methods comprise creating a phase contrast image of skin tissue and examining the tissue to assess at least one of the presence and the absence of at least one indicator of a disease. In some embodiments the methods comprise creating a phase contrast image of brain tissue and examining the tissue to assess tissue morphology. In some embodiments the methods comprise creating a phase contrast video of blood flow to assess blood flow velocity. In some embodiments the methods comprise creating a phase contrast video of blood flow to assess the cell count of at least one blood cell type.

In another aspect this disclosure provides an apparatus for creating a phase contrast image of a sample, comprising: a probe comprising 1) an optical radiation source or a first light conduit, and 2) a photo detector array or image conduit, and 3) a distal end; wherein the light conduit, the photo detector array or image conduit, and the distal end of the probe are configured to back illuminate the target region of a sample in contact or near contact with the distal end of the probe with a light from the first light source to provide a first oblique back illumination of the target region of the sample, and to detect a first phase contrast image from light originating from the first light source and back illuminating the target region of the sample.

In some embodiments the distal end of the optical radiation source or first light conduit extend to the distal end of the probe. In some embodiments the distal end of the optical radiation source or first light conduit is recessed from the distal end of the probe by up to 10 cm. In some embodiments distal end of the photo detector array or image conduit is recessed from the distal end of the probe.

In some embodiments the probe comprises a first light conduit and the apparatus further comprises a first optical radiation source connected to or projected to a proximal end of the first light conduit. In some embodiments the probe comprises a photo detector array. In some embodiments the probe comprises an image conduit and a proximal end of the image conduit is connected to or imaged to a photo detector array.

In some embodiments the probe further comprises a second optical radiation source or a second light conduit; wherein the second optical radiation source or second light conduit, the photo detector array or image conduit, and the distal end of the probe are configured to illuminate the target region of a sample in contact or near contact with the distal end of the probe with a light from the second light source to provide a second oblique back illumination of the target region of the sample, and to detect a second phase contrast image from light originating from the second light source and back illuminating the target region of the sample.

In some embodiments the distal end of the optical radiation source or first light conduit extends to the distal end of the probe. In some embodiments the distal end of the optical radiation source or first light conduit is recessed from the distal end of the probe by up to 10 cm. In some embodiments the distal end of the photo detector array or image conduit is recessed from the distal end of the probe.

In some embodiments the probe comprises a second light conduit and the apparatus further comprises a second optical radiation source connected to or imaged to a proximal end of the first light conduit.

In some embodiments the apparatus comprises at least three optical radiation sources or a light conduits, wherein the at least three optical illumination sources or light conduits are located at distinct locations around the probe such that each is capable of creating oblique back illumination enabling the measurement and display of phase gradients in different directions relative to the others.

In some embodiments the axis of illumination of the sample with the first light source and the axis of detection of light originating from the first light source and back illuminating the target region of the sample are different. In some embodiments the axis of illumination of the sample with the second light source and the axis of detection of light originating from the second light source and back illuminating the target region of the sample are different.

In some embodiments the axis of detection of light originating from the first light source and reflected from the sample and the axis of detection of light originating from the second light source and illuminating the target region of the sample are different. In some embodiments the axis of detection of light originating from the first light source and reflected from the sample and the axis of detection of light originating from the second light source and back illuminating the target region of the sample are the same. In some embodiments the wavelength of the light from the first light source and the wavelength of light from the second light source are different.

In some embodiments the apparatus is configured to detect the first and second images during first and second non-overlapping time intervals. In some embodiments the apparatus is configured to detect the first and second images during first and second overlapping time intervals. In some embodiments the apparatus is configured for illumination of the sample by the first and second light sources with light of different distinguishable wavelengths. In some embodiments the apparatus is configured for illumination of the sample by the first and second light sources with orthogonally polarized light.

In some embodiments the first and second light sources are capable of providing illumination at a range of wavelengths comprising from 0.2 to 300 μm.

In some embodiments the light source is selected from a light-emitting diode (LED), a laser, a supercontinuum light source, or a superluminescent diode (SLED). In some embodiments the apparatus comprises a photo detector array. In some embodiments the photo detector array is a charge coupled device (CCD) or a CMOS (complementary metal oxide semiconductor) camera sensor.

In some embodiments the apparatus comprises an optical conduit to communicate light in at least one direction selected from toward the sample and away from the sample.

In some embodiments the apparatus is configured so that the axis of illumination of the sample with the first light source and the axis of detection of light originating from the first light source are displaceable by from about 0.2 mm to about 5 mm. In some embodiments the apparatus is configured so that the axis of illumination of the sample with the second light source and the axis of detection of light originating from the second light source are displaceable by from about 0.2 mm to about 5 mm.

In some embodiments the apparatus is configured to obtain images of object planes of the target region from the surface of the sample to about 300 μm below the surface of the sample. In some embodiments the apparatus creates images laterally resolved at from about 0.3 μm to about 10 μm.

In some embodiments at least one of 1) the distal end of the first optical radiation source or first light conduit, 2) the distal end of the second optical radiation source or second light conduit, and 3) the distal end of the photo detector array or image conduit, extend through and end at the distal end of the probe.

In some embodiments at least one of 1) the distal end of the first optical radiation source or first light conduit, and 2) the distal end of the second optical radiation source or second light conduit, and 3) the distal end of the photo detector array or image conduit, is recessed from the distal end of the probe by up to 5 cm.

In another aspect an endoscope that comprises an apparatus of this disclosure is provided. In some embodiments the endoscope is portable.

In another aspect this disclosure provides a system that comprises an apparatus of this disclosure and a processor for processing images obtained from the apparatus. In some embodiments the system comprises an endoscope that comprises an apparatus of this disclosure.

In another aspect this disclosure provides methods of creating at least one of a phase contrast image of a target region of a sample and a difference image of two phase contrast images of a target region of a sample, comprising: providing a sample comprising a target region; using an apparatus of this disclosure to create at least one phase contrast image of the target region of the sample using a method of this disclosure, and optionally creating a difference image from the two or more contrast images of the target region of the sample.

In another aspect this disclosure provides a phase contrast image created by a method of this disclosure. Also provided is a data set representing the phase contrast image.

In some embodiments the phase contrast image is stored on a tangible computer-readable medium or machine-readable medium. Such media include, for example, hard disks, removable magnetic disks, removable optical disks (e.g., compact disks and digital video disks), magnetic cassettes, memory cards or sticks, random access memories (RAMs), read only memories (ROMs), and the like.

### BRIEF DESCRIPTION OF THE DRAWINGS

FIG. 1a shows the principle of OBM. Illumination is launched into tissue via an offset fiber. Multiply scattered light leads to oblique trans-illumination of object plane (dashed).

FIG. 1b shows the principle of OBM. Alternating illumination through two fibers, and imaging through flexible endoscope. 201 is a sample, the hatched line is the target region, 301 is an image conduit, 321 and 322 are optical elements in the image conduit, 101 and 102 are light conduits, 111 and 112 are LEDs, 401 is a camera.

FIG. 1c shows that oblique illumination leads to phase gradient contrast. With no phase gradient, oblique illumination is partially blocked by aperture in detection optics.

FIG. 1d shows that oblique illumination leads to phase gradient contrast. Phase gradients due to slopes in refractive index variations can lead to more blockage.

FIG. 1e shows that oblique illumination leads to phase gradient contrast. Phase gradients due to slopes in refractive index variations can lead to less blockage.

FIG. 2 shows the principle of OBM. Illumination is launched into tissue via an offset fiber. Multiply scattered light leads to oblique trans-illumination of object plane (dashed).

FIG. 3 shows OBM images of onion skin. (a) and (b) are raw images acquired with left and right illumination. (c) is absorption image (a+b). (d) is a phase gradient image (a-b). Note: panel (d) contains negative values, meaning its zero level is gray. Scale bar=100 μm. Depth=70 μm.

FIG. 4a shows an OCT image.

FIG. 4a shows a representative reflected light path that generates the OCT image.

FIG. 4c shows a DIC image.

FIG. 4d shows a representative transmitted light path that generates the DIC image.

FIG. 5 shows an illumination module. In this embodiment computer-controlled LEDs can span from UV to NIR.

FIG. 6a shows a lateral view of an embodiment of distal illumination optics. 601 is a probe, 301 is an image conduit that extends to the distal end of the probe (hidden from view), 101 and 102 are first and second light conduits.

FIG. 6b shows a cross sectional view of an embodiment of distal illumination optics comprising a molded fiber end.

FIG. 6c shows a cross sectional view of an embodiment of distal illumination optics comprising multiple thin fibers. In this case the first light conduit comprises five light conduits (101) and the second light conduit comprises five light conduits (102). Each set of light conduits is located on opposite sides of the distal probe and thus each set acts as a light conduit to provide oblique back illumination of the target region of a sample.

FIG. 7a shows an example of single shot OBM using polarization discrimination.

FIG. 7b shows an example of single shot OBM using spectral discrimination.

FIG. 7c shows an example of single shot OBM using polarization discrimination with the addition of polarizers in the aperture plane.

FIG. 7d shows an example of single shot OBM using spectral discrimination with the addition of spectral bandpass filters in the aperture plane.

FIG. 8a shows a Monte Carlo simulation of the “photon banana”.

FIG. 8b shows a Monte Carlo simulation of photon exit angles.

FIG. 8c shows additional Monte Carlo simulations of photon exit angles.

FIG. 8d shows estimates of the dependence of mean and median exit angles and detected intensity as a function of fiber-probe separation.

FIG. 9 shows OBM images of ex-vivo rat colon, acquired with 530 nm LEDs. (a) and (b) are images acquired with left and right illumination after core removal (zoomed insets are before core removal). Exposure time per image is 2 ms. (c) is sum image. (d) is difference image. Note: panel (d) contains negative values, meaning its zero level is gray. Field of view=100 μm. Depth=70 μm. Resolution=6 μm.

FIG. 10 shows OBM images of various regions of ex-vivo rat intestine, acquired with 530 nm LEDs. Top row is sum images; bottom row is difference (phase gradient) images. Panels (a)-(d) show small intestine (note high resolution content in panel (b); panel (d) highlights epithelial villi). Panels (e)-(h) show large intestine (panel (f) highlights a crease in tissue; panel (h) presumably highlights a blood vessel). Note much higher contrast of phase gradient images compared to relative featurelessness of absorption images. Field of view=600 μm. Depth=70 μm.

FIG. 11a and FIG. 11b are OBM phase-gradient images of mouse small-intestine villi (same sample as in FIG. 10) acquired with Hopkins rod-lens. FIELD OF VIEW is 500 μm. Working distance is 50 μm. A and B are two frames from a movie taken while focus depth was being adjusted. Green LED illumination is delivered through two diametrically opposed 1 mm fibers.

FIG. 12 shows (a) a phase-gradient image of a 45 μm bead embedded in agarose made scattering by 2 μm beads (also apparent, demonstrating resolution), (b) phase gradient appears linear. Simultaneous (c) amplitude and (d) phase-gradient images of blood flow in chick embryo (movement in (c) is highlighted in (d)); scale bar=75 μm. Simultaneous (e) amplitude and (f) phase-gradient images acquired with higher magnification fiber bundle; scale bar=30 μm. (g) and (h) are high magnification phase-gradient images when blood flow is slowed (Note different shapes of blood cells); scale bar=30 μm.

FIG. 13 shows amplitude (top) and phase-gradient (bottom) mosaic of 300 frames acquired at 17.3 frames per second while manually scanning across a chick embryo. Scale bar=75 μm. Box indicates single frame size (186×139 μm).

FIG. 14 shows a labeled version of FIG. 12d. Visible morphological structures include (l) crypt lumens, (c) epithelial cells, (ap) apical border of epithelial cells, (bl) basolateral border of epithelial cells, and (lp) lamina propia.

FIG. 15 shows optical biopsy results. Panels (a-c) are images of (a) large, (b) mid and (c) small intestine regions. Note villi in panel (c) (FIELD OF VIEW=600 μm). Panels (d) and (e) are images of (d) large, and (e) small intestine at higher magnification (Field of view=240 μm). Note crypts in panel (d).

FIG. 16 shows a comparison of added versus subtracted raw OBM images of a 45 μm polystyrene bead. Raw images under oblique back-illumination from two opposing directions are shown in FIGS. 16(a) and 16(b). Addition of (a) and (b) cancels phase gradient contrast and emphasizes absorption, as demonstrated in FIG. 16(c). Subtraction of (a) and (b) cancels absorption contrast and emphasizes phase gradients, as shown in FIG. 16(d).

FIG. 17 shows a demonstration of apparent axial resolution of OBM.

FIG. 18 shows images of mouse lung and liver. Fixed mouse liver imaged under phase gradient contrast OBM (a) and (b). Fixed mouse lung cross-section imaged under phase gradient contrast OBM (c) and (d).

FIG. 19 shows images of rat flank skin. (a) is an en face view 50 μm below the surface showing wavy collagen strands. (b) is an en face view 100 μm below the surface showing a cluster of adipocytes (stars).

FIGS. 20 shows OBM penetration depth.

FIG. 21 shows shows OBM penetration depth.

FIG. 22 shows a comparison of OBM with epi-illumination reflection contrast.

FIG. 23 (a-c) shows a dual-camera, multi-wavelength setup.

FIG. 24 shows OBM using a single illumination wavelength. (a) and (b) show individual phase gradient contrast OBM images under simultaneous red and NIR illumination. (c) shows a capillary visualized by a sliding 3-frame temporal variance filter. Multiplying frames in FIG. 24(a, and b) by capillary mask FIG. 24(c) yields the images in FIGS. 24(d, and e), where individual red blood cells are easily distinguished (white arrows). FIG. 24(f) shows another capillary with a more tortuous path extracted from a separate segment from the same video as FIGS. 24(a-e).

FIG. 25 shows simultaneous co-registered multi-wavelength OBM. Phase gradient (a and c) images and corresponding amplitude images (b and d) were obtained under red and blue/green illumination, respectively.

FIG. 26 shows schematics of different OBM illumination configurations. (a) illustrates llumination delivered via fiber optic conduits in contact with the tissue surface. (b) illustrates illumination delivered by light sources not in contact with the tissue surface.

FIG. 27 shows a comparison of OBM with fiber-mediated illumination in contact with the tissue versus non-fiber-mediated illumination not in contact with the tissue. (a) and (b) are OBM images acquired with fiber-delivered LED light (˜650nm). (c) and (d) are OBM images acquired with a 6-element LED flashlight held approximately 2 inches from the sample surface.

FIG. 28 shows a design of OBM based on illumination and detection through a common microscope objective.

FIG. 29 shows a design of miniature OBM endomicroscope probe.

### DETAILED DESCRIPTION

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. Generally, nomenclatures used in connection with microscopy, imaging and endoscopy, described herein are those well-known and commonly used in the art. Certain references and other documents cited herein are expressly incorporated herein by reference. In case of conflict, the present specification, including definitions, will control. The materials, methods, and examples are illustrative only and not intended to be limiting.

The methods and techniques of the present disclosure are generally performed according to conventional methods well known in the art and as described in various general and more specific references that are cited and discussed throughout the present specification unless otherwise indicated. See, e.g., L. V. Wang and H.-i Wu, Biomedical Optics: Principles and Imaging, 1st ed. Wiley-Interscience, 2007; J. C. Mertz, Introduction to Optical Microscopy, Roberts and Company Publishers, 2009.

Before the present devices, systems, methods, and other embodiments are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “comprising” as used herein is synonymous with “including” or “containing”, and is inclusive or open-ended and does not exclude additional, unrecited members, elements or method steps.

This disclosure provides a new phase contrast technique that works in a reflected light geometry and is thus amenable to use on tissues that for one or more reasons are not amenable to transmission lighting. One non-limiting example is endoscopy applications. This method is sometimes referred to herein as “oblique back-illumination microscopy, or “OBM”. OBM requires no tissue labeling and provides high resolution differential interference contrast (DIC)-like images of sub-surface sample morphology in an epi-detection configuration.

In some embodiments the methods, apparatus, and systems provided herein can be used for optical biopsies of tissue.

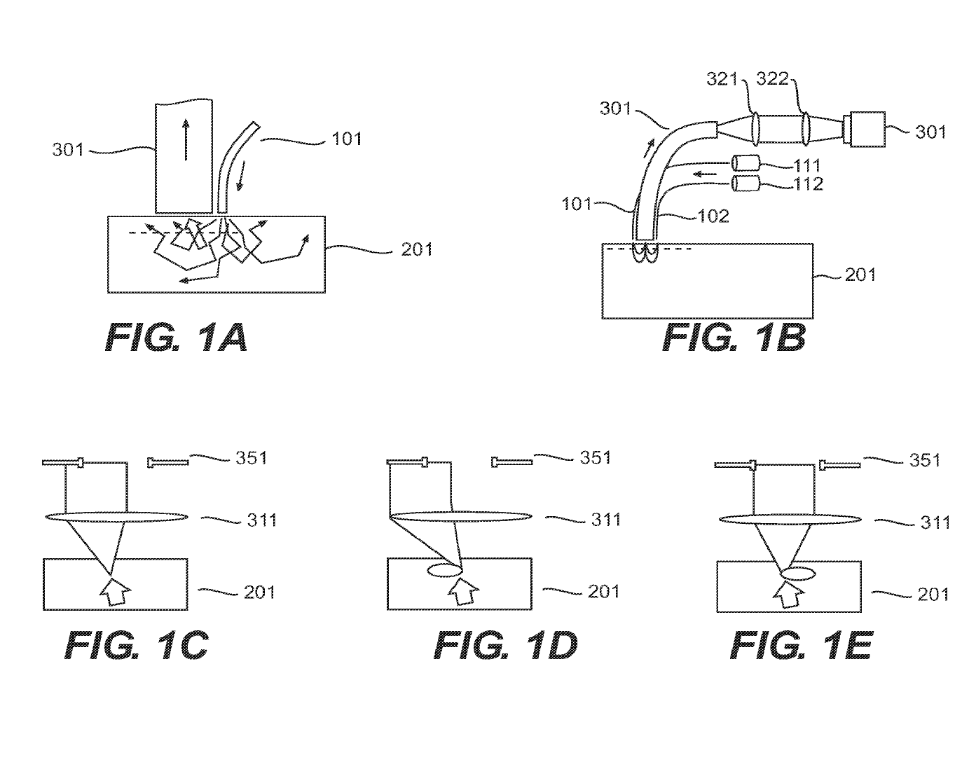

In certain embodiments OBM uses standard wide field detection optics. That is, light is projected from an object plane to an image plane with a series of lenses, and it is then detected with a camera (for example, a CCD or CMOS). In the case of an endoscope, some extra relay optics may be introduced, such as an imaging fiber bundle or a Hopkins rod-lens. This is not necessarily any different than standard wide field endoscopy. Where OBM differs is in the illumination path. A schematic is shown in FIG. 1a, depicting the distal end of an OBM endoscope. As illustrated, the illumination is launched into the tissue sample (201) via an off-axis optical fiber (101) (or a pair, as shown in FIG. 1b (101 and 102)). Microscopic resolution endoscopes (or endomicroscopes) are invariably contact mode, or near contact mode. In such configurations, illumination light reflected directly from the sample surface is not detectable (this can be further ensured by, e.g., recessing the illumination fibers a bit).

The only light that is detected is illumination light that has been multiply scattered to such a degree that it is re-directed toward the sample surface and incident upon a photo detector array or image conduit (301). In this manner, the object plane (defined as the plane that is in focus with respect to the detection optics, and indicated by a hatched line in the FIG. 1a), is back-illuminated. A critical point to note here is that, because the illumination source is off-axis, the back-illumination flux at the object plane is directed, on average, not quite vertically but with a slight tilt away from the illumination source. That is, the back-illumination is oblique, which results in OBM in this configuration.

Oblique illumination has been used in other contexts to obtain phase contrast, or, more precisely, phase gradient contrast. For example, the simple misalignment of the condenser in a standard transmission wide field microscope leads to phase gradient contrast. The reason for this can be understood intuitively from FIGS. 1c-1e. Oblique illumination locally originating from the object plane is partially blocked by the detection aperture (351). The presence of a local phase slope (gradient) (221) in the object (201) refracts the illumination one way or the other, depending on the sign of the slope, thereby leading to a respective increase or decrease in the detected intensity. Thus, phase slopes are converted to intensity variations of related sign, yielding phase gradient contrast imaging. As shown schematically in FIGS. 1c-1e, the aperture in the detection optics plays a role in creating phase gradient contrast images. Indeed, the inventors have shown in other contexts that phase gradient contrast can be further enhanced by combining oblique illumination with oblique detection [36]. Accordingly, in some embodiments of the methods and apparatus of this disclosure oblique illumination and oblique detection are combined.

While illumination with a single off-axis source is enough to achieve phase-gradient contrast in an oblique back illumination configuration, the inventors have also discovered that a second off-axis source approximately diametrically opposed to the first (FIG. 1b) allows for certain useful variations on the technique. The phase gradient contrast resulting from the second source has opposite sign to that resulting from the first. The acquisition of two raw images, one from each source, yields many useful features to certain embodiments of the methods and apparatus disclosed herein, such as without limitation: 1) in some embodiments the difference image enhances phase gradient contrast and cancels any absorption contrast; 2) in some embodiments the sum image reveals absorption contrast and cancels any phase gradient contrast; 3) in some embodiments the difference image rejects uniform background, providing axial resolution; and 4) the use of dual illumination provides, in certain embodiments, a more homogenous net distribution of light in the processed images.

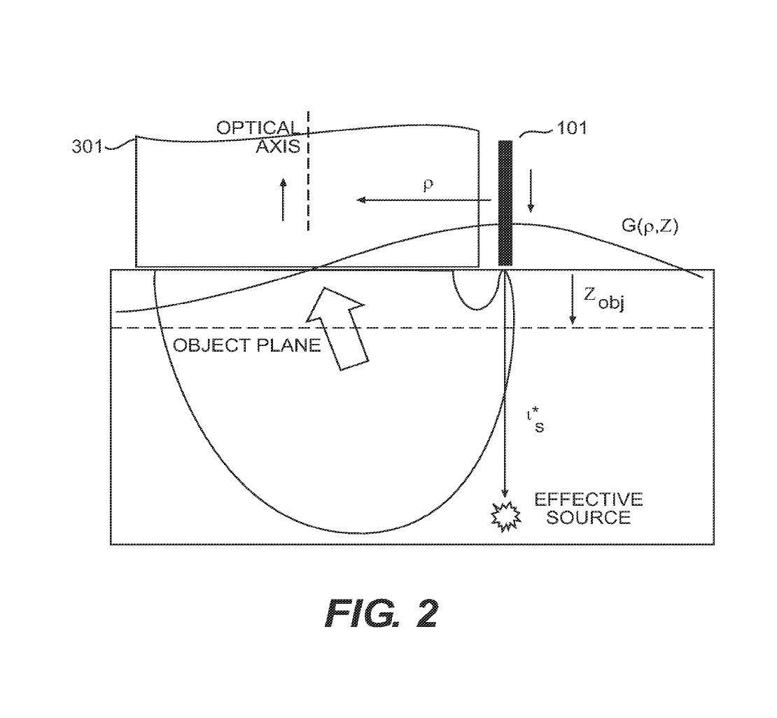

Certain geometrical parameters of the OBM technique are shown in FIG. 2. For simplicity, the figure only includes a single light source (101). This figure serves only as a schematic to depict how the illumination light can be roughly mimicked by a virtual light source a distance lₛ* below the physical light source. This approximation is reasonably valid for offset distances p on the order of lₛ* or greater.

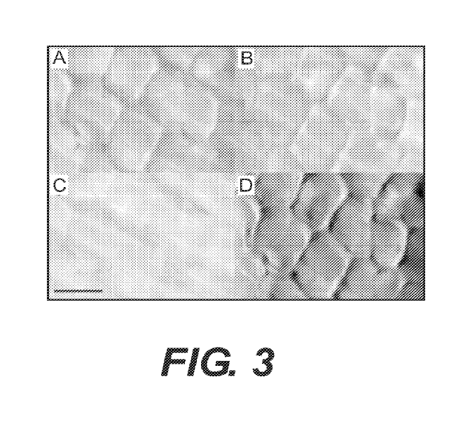

OBM embodiments that utilize two light sources are based on the acquisition of two images, with illumination states of opposing obliquity, as obtained, for example, when the illumination sources have equal but opposing offsets from the optical axis. It is believed that the back-illumination is not only oblique but also non-uniform in intensity (see FIG. 2). That is, regions of the image farther from the source will appear dimmer. To compensate for this intensity drop-off, as well as the possibility of non-equal source powers, the raw images are, in some embodiments, individually “flattened” and normalized (e.g. FIGS. 3a and 3b). This is done numerically prior to their subtraction or addition in embodiments in which images are combined. (Recall that image subtraction leads to phase gradient contrast, while image addition leads to absorption contrast.)

An useful property of the subtracted image is that it contains no uniform background. In fact, it contains no uniform signal at all, background or otherwise. Said differently, the difference image only contains non-zero spatial frequencies because it reveals phase derivatives instead of absolute phases. But by definition, non-zero lateral spatial frequencies in a standard microscope image are axially resolved [37]. That is, all the structure observed in FIG. 3d. must arise from regions of the sample that are (more or less) close to the focal plane (in this case, 70 μm deep). Because phase derivative images are intrinsically axially resolved, in some embodiments of the methods and apparatus of this disclosure the axial resolution is controlled. In some embodiments a particular axial resolution is specified by a user. In some embodiments high-pass spatial filters are used to provide tighter axial resolution (at the expense of low frequency image content). In some embodiments the types of filters, such as wavelets, are used.

OBM works in the reflection direction, like OCT. And yet, OBM images are not at all similar to OCT images. Instead, they are similar to DIC images, which are obtained in the transmission direction. Part of the reason for that is that OBM reveals phase gradients as opposed to absolute phase. But another more fundamental reason is that OBM is actually a transmission (i.e. trans-illumination) microscope in disguise.

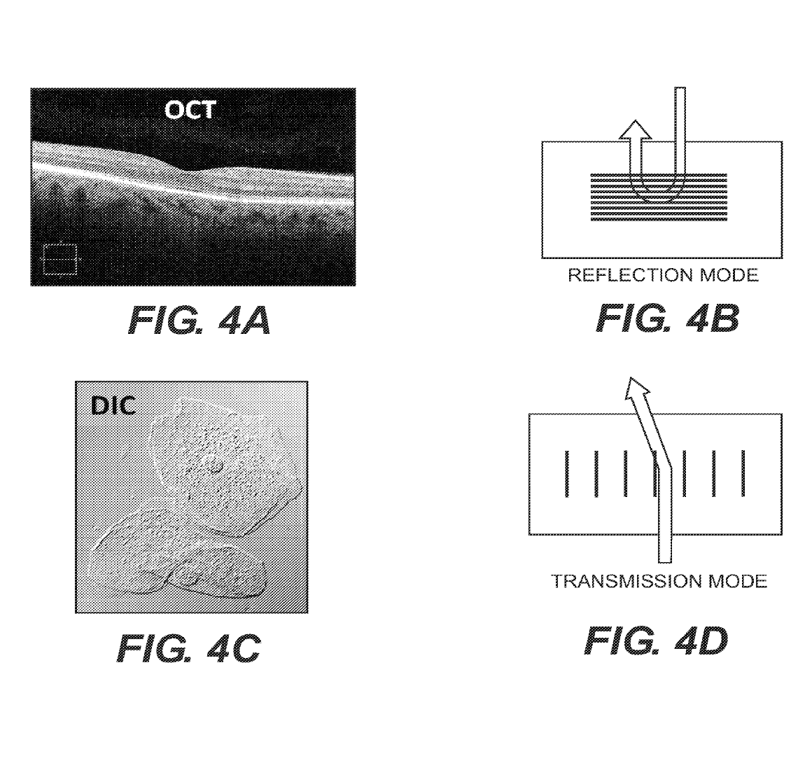

OCT is based on the detection of reflected light (shown schematically in FIG. 4b) whereas DIC is based on the detection of transmitted light (shown schematically in FIG. 4d). For a tissue to cause reflection, it must exhibit index of refraction variations with very high axial spatial frequency (at least as high as 2k, where k=2π/λ is the light wavenumber and X the light wavelength in the medium). In contrast, for a tissue to deflect a beam by a small angle θ (less than 1), it need only exhibit lateral index of refraction spatial frequencies on the order kθ. Thus, OCT reveals large axial spatial frequencies whereas DIC reveals much smaller lateral spatial frequencies. These differences are demonstrated by the images in FIG. 4a (OCT) and 4c (a DIC image that is similar in image quality to OBM).

There are several useful consequences to working in the trans-illumination direction (which explains the popularity of transmission-based optical microscopes). The main advantage is that light scattering in tissue is dominantly forward directed, because scattering structures in tissue are typically micron scale or larger [40]. As noted, to obtain direct backward scattering from tissue requires structures that exhibit very high axial spatial frequencies, such as abrupt interfaces or tiny punctate scatterers. OCT beautifully reveals such high axial-frequency structures, but it cannot reveal more subtle, lower frequency features that are prevalent in tissue. In contrast, trans-illumination microscopes, since they are sensitive to even minute deflections of light, do reveal low frequency features (in addition to high frequency features). This makes OBM very useful and enables its use, in some embodiments, as an effective tool for assessing tissue pathology. Indeed, the differences between healthy and diseased tissue are often very subtle.

Several parameters can affect signal to noise ratio (SNR), including shot noise. For example, OBM embodiments that combine images involve subtracting or adding two images of roughly equal intensity. In both cases, the shot noise in the final image is increased by a factor of the square root of 2. To maximize the SNR associated with shot noise one need simply maximize the amount of detected light. OBM is not a fluorescence technique, so scattered light is plentiful. Even for very short exposure times (˜1 millisecond), a camera used to detect images generated with OBM can be operated close to saturation. Camera readout and dark noise play essentially no role in this regime. In some embodiments, camera pixel well capacity is as large as feasible. In other words, high-end scientific cameras designed for fluorescence imaging are not ideal for some embodiments. Instead, what is most useful for such embodiments are simple machine-vision cameras. In addition to featuring large well capacities (>10⁵ e-), these offer additional benefits of high speed (>100 fps), and low cost. An example currently on the market is cameras manufactured by Photonfocus AG.

In some embodiments of the methods and apparatus disclosed herein, real-time image information is captured at a near video rate. As described above, OBM is a two-shot system, meaning the maximum OBM frame rate is half the camera frame rate. Machine vision speeds easily satisfy real-time criteria; however, if there is a time delay between the two shots, and if the tissue (or probe) is rapidly moving or changing somehow, then motion artifacts could occur. Thus, in some embodiments a double-shutter camera (e.g. Pixelfy, Cooke Corp.) is used, which acquires images pairwise, with essentially zero inter-pair frame delay (<5 μs). To reduce motion during the frame exposures fast exposures (and a lot of light) are used in some embodiments. In some embodiments the two exposures are merged into a single exposure.

In some embodiments of the two-shot technique, the shots are discriminated by time. In alternative embodiments other parameters are used to discriminate the shots, such as wavelength or polarization. In the former case, the left and right illumination sources provide light of different colors, allowing a spectral separation within a same camera frame, for example. In the latter case, the left and right illumination sources are orthogonally polarized. The inventors have experimentally verified that polarization is partially preserved even after multiple scattering in a retro-reflection geometry (provided length scales are not too long), in agreement with previous studies [42-45]. This demonstrates the feasibility of polarization-based separation of both shots within the same camera frame.

It is believed that OBM will be particularly useful in endoscope configurations. However, in some embodiments OBM is deployed in a freestanding microscope configuration, for example. In some embodiments the OBM endoscope is a portable, standalone device. In some embodiments a fiber bundle is used to collect and relay the image to a photo detector array (or camera sensor).

In some embodiments, uneven spacing of the fiber cores of the image conduit causes raw images to appear corrupted by irregular sampling. This can be addressed with a fast image processing algorithm to very effectively remove these core-spacing related artifacts. The algorithm is based on a nonlinear, iterative segmentation-interpolation strategy that maintains high spatial resolution (described in detail in [35]). This algorithm, along with the two-shot triggering and data-transfer protocols necessary to operate HiLo microscopy are already coded in CUDA to run on a graphical processing unit (GPU). Reference 35 is hereby incorporated herein by reference. In some embodiments these are incorporated into an OBM system, apparatus, or method of this disclosure.

In some embodiments OBM is operated with LED illumination. This is useful for several embodiments. LEDs are inexpensive, robust, available in a variety of wavelengths, and can be rapidly turned on and off (30 kHz measured). Moreover, they are spatiotemporally incoherent, meaning they do not produce speckle. However, incoherence also has a drawback. Incoherent light, which occupies a large “phase space”, cannot be compressed (focused) into the small phase-space of a fiber without incurring significant power loss. Based on simple étendue arguments [37], the maximum coupling efficiency of LED light into a fiber is given by roughly NA²_{fiber}(A_{fiber}/A_{LED}), where NA_{fiber} and A_{fiber} are the fiber NA and area, and A_{LED} is the LED area. To accommodate this, in some embodiments a large area, multimode fiber is used. For example, in some embodiments a 400 mW Luxeon LED coupled into a 1000 682 m fiber core delivers almost 30 mW.

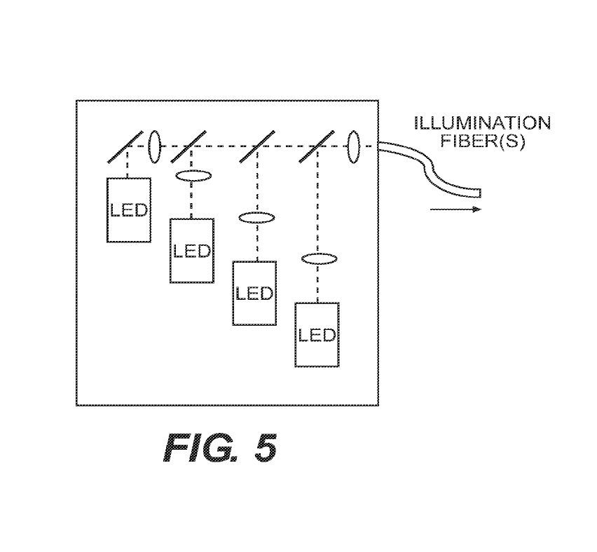

In some embodiments the LED(s) are housed in a module. For enhanced versatility with respect to wavelength in some embodiments the our module will houses several different color LEDs (see FIG. 5), allowing the user to select different colors or perform multicolor imaging. The design of this module is similar in concept to the Zeiss Colibri™, with the difference that the LEDs are coupled into an optical fiber rather than a light pipe. In some embodiments two of these modules are used for a single OBM apparatus of this disclosure (left/right illumination). In some embodiments the control of the LEDs can be both analog (power) and digital (on/off). In some embodiments the housing can accept different size fibers via a standard interconnect.

In some embodiments the imaging fiber bundle, with its miniaturized distal imaging optics, is threaded through the accessory port of a probe, such as a standard flexible colonoscope. In some embodiments the diameter of this port is about 3.2 mm. In some embodiments, the diameter of the distal optics is 2.8 mm.

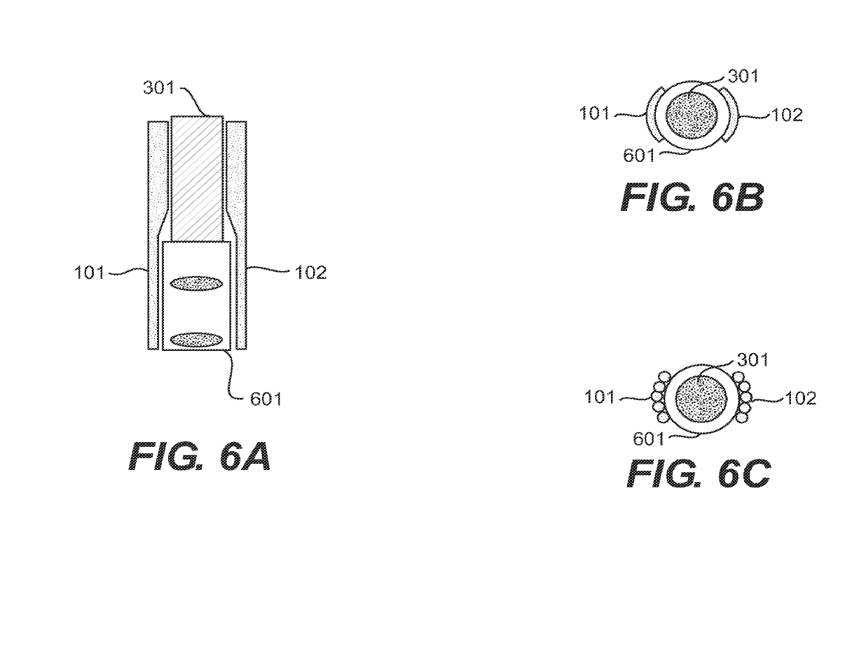

Some representative non-limiting examples of the distal end of an endoscope configuration of OBM are shown in FIG. 6. FIG. 6a is a lateral view, showing the distal end of the probe (601), two illumination optical fibers (101 and 102), and a light conduit (301). In one embodiment, graded-index plastic optical fibers (Thorlabs or other), are heat-molded at their distal end into a more space conserving geometry (sufficient A_{fiber} is maintained for adequate throughput) (FIG. 6b). As alternative plan, multiple thinner fibers arranged in arcs at the distal end and circular bundles at the proximal end are used (FIG. 6c). Finally, an intriguing possibility is to incorporate mini LEDs directly into the endoscope housing itself. This would require connecting with electrical wires, but, in the end, may deliver the most optical power per utilized space. No heat sinks are necessary since the power requirements are very modest.

In some embodiments, lasers are used as the illumination source. These can deliver more power into thin optical fibers than LEDs, however they have the disadvantage of producing speckle, thereby possibly leading to image granularity. In other embodiments, superluminescent diodes (SLED) are used as the illumination source These are similar to lasers in that they can deliver more power into thin optical fibers than LEDs. Because they produce no speckle they can be preferable to lasers in certain embodiments.

A characteristic of OBM is that the illumination can be decoupled from the detection optics, such that the illumination does not go through the detection optics, as it does in many epi-imaging devices. This is useful because it avoids spurious back-reflections from glass interfaces, etc. It also makes extended image relay optics unnecessary in certain embodiments. Thus, in some embodiments a proximal camera is used in the OBM apparatus.

In other embodiments the proximal camera is replaced by a miniaturized distal camera, such as by way of example one mounted directly at the end of the endoscope. Thus, in some embodiments the apparatus comprises an all-electric coupled distal end (illumination and detection).

Depending on their application, endoscopes can be flexible or rigid. Rigid endoscopes can be larger than flexible endoscopes—up to several millimeters in diameter. In some embodiments of a rigid endoscope the length scales involved are larger and longer illumination wavelengths are used. Fortunately, near infra-red LEDs are readily available.

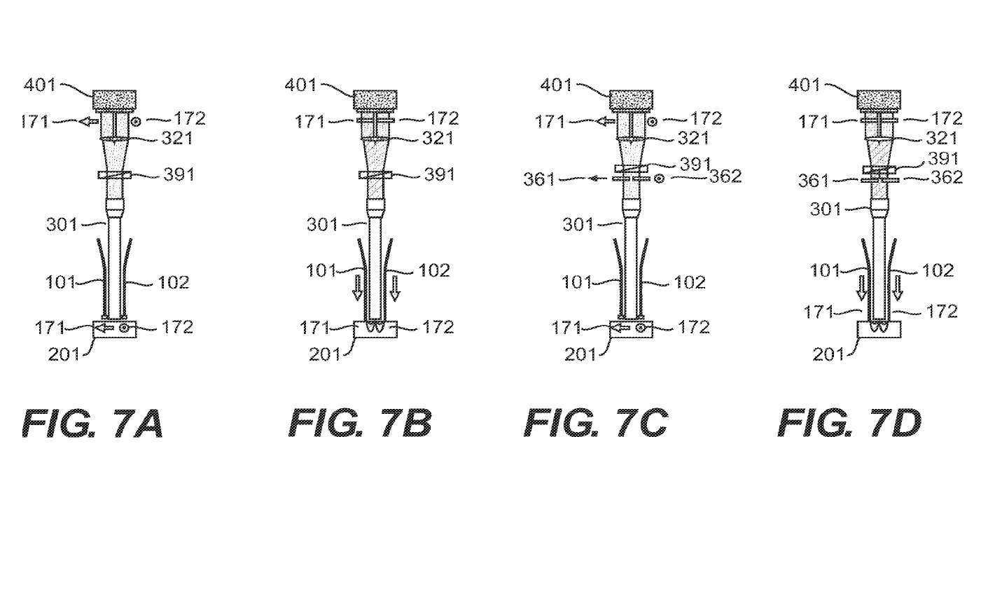

A key source of usefulness of a rigid Hopkins-type endoscope is that, because it is based on simple lenses and free-space propagation, optical phase is preserved from the object plane to the detector plane. Moreover, the aperture plane can be accessed and in some embodiments oblique illumination is combined with complementary oblique detection [36]. In some embodiments oblique detection is achieved by introducing beam half-blocks in the detection aperture plane, and switching sides depending on which LED is illuminated. In some embodiments this is done in a single shot and with no moving parts. Two exemplary strategies for this are illustrated in FIG. 7. In both strategies, a Wollaston prism (or other beam separating mechanism) is placed at the aperture plane (391), which splits the beam in two and projects the two images simultaneously onto the camera. In the case of polarization discrimination (FIG. 7a), simultaneous cross-polarized illumination is used (as noted above, polarization is partially maintained in tissue). In the case of spectral discrimination (FIG. 7b), simultaneous two-color illumination, which is assumed to be randomly polarized is used. Half-aperture blocks can then be inserted, either in the form of polarizers (FIG. 7c) or spectral bandpass filters (FIG. 7d), to confer some degree of obliqueness to the detection. It should be noted that, while oblique detection enhances phase gradient contrast, it degrades absorption contrast. Depending on how important the latter feature is in any particular embodiment, full oblique detection can or cannot be used as the user chooses.

This disclosure also provides methods of creating a phase contrast image. In some embodiments the method comprises illuminating the target region of a sample with a first light source to provide a first oblique back illumination of the target region of the sample, and detecting a first phase contrast image from light originating from the first light source and back illuminating the target region of the sample.

As used herein a “target region” is the portion of a sample that from which an image is desired. Alternatively, the “target region” is the portion of the sample from which an image is created and/or captured.

As used herein, “oblique back illumination” means illumination that results from the re-direction of light into the backward direction by a multiple scattering process within a tissue. Oblique back illumination is created by an off-axis illumination source. As a result, the back-illumination flux at the object plane is directed, on average, not quite perpendicular to the plane but with a slight tilt away from the illumination source. Oblique back illumination may be created with light sources that are in contact with a sample. Oblique back illumination may also be created by light sources that are not in contact with the sample. In some embodiments one or more of each type of light source are combined in an apparatus or used in a method.

In some embodiments of the method, the method further comprises illuminating the sample with a second light source to provide a second oblique back illumination of the target region of the sample, and detecting a second phase contrast image from light originating from the second light source and back illuminating the target region of the sample. In some embodiments the method further comprises creating a difference contrast image of the target region of the sample by subtracting the second phase contrast image of the target region of the sample from the first phase contrast image of the target region of the sample. In some embodiments the method further comprises creating an absorption contrast image of the target region of the sample by adding the first phase contrast image of the target region of the sample to the second phase contrast image of the target region of the sample. In some embodiments the method the difference contrast image and the absorption contrast image are analyzed together to infer at least one property of the sample. In some embodiments one of the difference contrast image and the absorption contrast image is analyzed in a way that the other is not in order to infer at least one property of the sample.

In some embodiments of the method, the axis of illumination of the sample with the first light source and the axis of detection of light originating from the first light source and back illuminating the target region of the sample are different. That is, the light source is off axis, meaning among other things that it is delivered independently of the detection optics. In some embodiments of the method, the axis of illumination of the sample with the second light source and the axis of detection of light originating from the second light source and back illuminating the target region of the sample are different. In some embodiments of the method, the axis of detection of light originating from the first light source and back illuminating the target region of the sample, the axis of detection of light originating from the second light source back illuminating the target region of the sample are different. Note that in such embodiments the illumination and the detection are both oblique. In some embodiments of the method, the axis of detection of light originating from the first light source and back illuminating the target region of the sample and the axis of detection of light originating from the second light source and back illuminating the target region of the sample are the same.

In some embodiments of the method, the first and second light sources illuminate the sample with light of different distinguishable wavelengths. In some embodiments of the method, the first and second light sources illuminate the sample with distinguishable orthogonally polarized light. In both of these types of embodiments it is possible to detect light from the first and second light sources simultaneously, although the method need not be conducted that way.

In some embodiments the method comprises detecting the first and second images during first and second non-overlapping time intervals. In such embodiments the wavelength of light from the first and second light sources can be (but need not be) the same.

In some embodiments of the method, the wavelength of the light from at least one of the first and second light sources is from 0.2 to 300 μm, from 0.2 to 1 μm, from 0.4 to 0.7 μm, from 0.2 to 0.3 μm, from 0.3 to 0.4 μm, from 0.4 to 0.5 μm, from 0.5 to 0.6 μm, or from 0.6 to 0.7 μm. In some embodiments of the method, the light source is selected from a light-emitting diode (LED), a laser, or a superluminescent diode (SLED). In some embodiments of the method, the detecting is by a photo detector array. In some embodiments of the method, the photo detector array is a charge coupled device (CCD) or a CMOS (complementary metal oxide semiconductor) camera sensor. In some embodiments the method comprises using an optical conduit to communicate light in at least one direction selected from toward the sample and away from the sample.

In some embodiments of the method, the axis of illumination of the sample with the first light source and the axis of detection of light originating from the first light source are displaced by from about 0.2 mm to about 3 mm, from about 0.5 mm to about 2.5 mm, from about 1 mm to about 2 mm, from about 1.5 mm to about 2.5 mm, or from about 2 mm to about 3 mm. In some embodiments axis of illumination of the sample with the first light source and the axis of detection of light originating from the first light source are displaced by about 0.2 mm, about 0.3 mm, about 0.4 mm, about 0.5 mm, about 1.0 mm, about 1.5 mm, about 1.75 mm, about 2.0 mm, about 2.25 mm, about 2.5 mm, about 3.0 mm, about 3.5 mm, about 4.0 mm, or about 5.0 mm. In some embodiments of the method, the axis of illumination of the sample with the second light source and the axis of detection of light originating from the second light source are displaced by from about 0.2 mm to about 3 mm, from about 0.5 mm to about 2.5 mm, from about 1 mm to about 2 mm, from about 1.5 mm to about 2.5 mm, or from about 2 mm to about 3 mm. In some embodiments axis of illumination of the sample with the second light source and the axis of detection of light originating from the second light source are displaced by about 0.2 mm, about 0.3 mm, about 0.4 mm, about 0.5 mm, about 1.0 mm, about 1.5 mm, about 1.75 mm, about 2.0 mm, about 2.25 mm, about 2.5 mm, about 3.0 mm, about 3.5 mm, about 4.0 mm, or about 5.0 mm. In some embodiments the displacement of the axis of illumination of the sample with the second light source and the axis of detection of light originating from the second light source, and the displacement of the axis of illumination of the sample with the first light source and the axis of detection of light originating from the first light source are the same. In some embodiments the displacement of the axis of illumination of the sample with the second light source and the axis of detection of light originating from the second light source, and the displacement of the axis of illumination of the sample with the first light source and the axis of detection of light originating from the first light source are different.

In some embodiments of the method, the object plane of the target region is from the sample surface to about 350 μm below the surface of the sample, from about 100 to about 300 μm below the surface of the sample, from about 150 to about 250 μm below the surface of the sample, from about 175 to about 225 μm below the surface of the sample. In some embodiments it is below the sample surface, greater than about 5 μm below the surface of the sample, greater than about 10 μm below the surface of the sample, greater than about 15 μm below the surface of the sample, greater than about 20 μm below the surface of the sample, greater than about 25 μm below the surface of the sample, greater than about 30 μm below the surface of the sample, greater than about 35 μm below the surface of the sample, greater than about 40 μm below the surface of the sample, greater than about 45 μm below the surface of the sample, greater than about 50 μm below the surface of the sample, greater than about 75 μm below the surface of the sample, greater than about 100 μm below the surface of the sample, greater than about 150 μm below the surface of the sample, greater than about 200 μm below the surface of the sample, greater than about 250 μm below the surface of the sample, greater than about 300 μm below the surface of the sample, or greater than about 350 μm below the surface of the sample.

In some embodiments of the method, the lateral resolution of the image is from about 0.3 μm to about 2 μm, the lateral resolution of the image is from about 1 μm to about 3 μm, the lateral resolution of the image is from about 2 μm to about 3 μm, the lateral resolution of the image is from about 2 μm to about 5 μm, the lateral resolution of the image is from about 2 μm to about 10 μm. In some embodiments of the method, the lateral resolution of the image is at least about 9 μm, the lateral resolution of the image is at least about 8 μm, the lateral resolution of the image is at least about 7 μm, the lateral resolution of the image is at least about 6 μm, the lateral resolution of the image is at least about 5 μm, the lateral resolution of the image is at least about 4 μm, the lateral resolution of the image is at least about 3 μm, the lateral resolution of the image is at least about 2 μm, the lateral resolution of the image is at least about 1 μm, the lateral resolution of the image is at least about 0.9 μm, the lateral resolution of the image is at least about 0.8 μm, the lateral resolution of the image is at least about 0.7 μm, the lateral resolution of the image is at least about 0.6 μm, the lateral resolution of the image is at least about 0.5 μm, the lateral resolution of the image is at least about 0.4 μm, or the lateral resolution of the image is at least about 0.3 μm.

In some embodiments of the method, the difference image is axially resolved.

In some embodiments the method further comprises obtaining a series of two or more images and combining the images to provide a composite image larger than the field of view a single image.

In some embodiments the method comprises creating a phase contrast image of gastrointestinal tissue and examining the tissue to assess at least one of the presence and the absence of indicators of a disease. In some embodiments the gastrointestinal tissue is colonic mucosa disease is at least one of hyperplasia and adenomatous changes.

Also provided herein are apparatus for creating a phase contrast image of a sample. In some embodiments the apparatus comprises a first light conduit, a photo detector array or an image conduit, and a distal end, wherein a distal end of the light conduit and the distal end of the photo detector array or image conduit extend to the distal end of the probe. In some embodiments the distal end of the light conduit and the distal end of the photo detector array or image conduit extend through and end at the distal end of the probe. In some embodiments at least one of the distal end of the light conduit and the distal end of the photo detector or image conduit is recessed from the distal end of the probe.

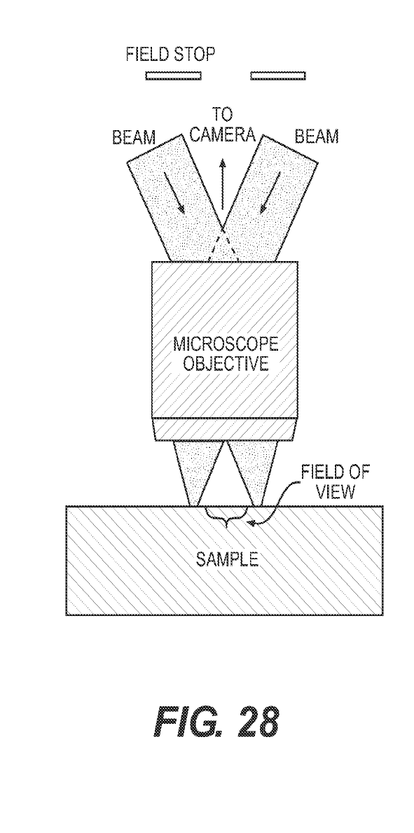

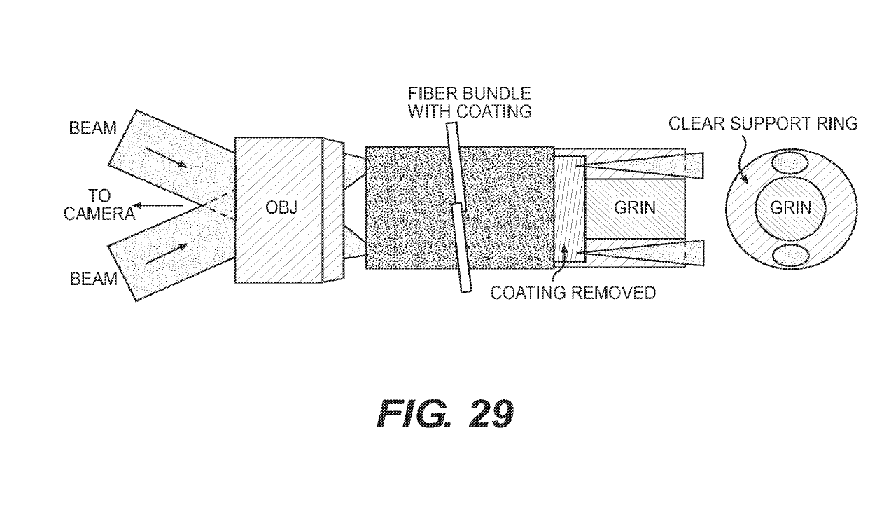

Two examples of OBM designed that use recessed illumination are shown in FIGS. 28 and 29. FIG. 28 shows a design of OBM based on illumination and detection through a common microscope objective. Collimated, oblique illumination beams are (roughly) focused to off-axis spots on the sample surface. Imaging of the sample is restricted by a field stop (somewhere in the detection optics) to a field of view that does not encompass the illumination spots. FIG. 29 shows a design of miniature OBM endomicroscope probe. Proximal end is shown on left, where two collimated, off-axis illumination beams are (roughly) focused into an imaging fiber bundle with an objective (OBJ). Distal end is shown on right, where a GRIN lens is optically cemented to the fiber bundle, centered by a clear support ring. Imaging is performed in the reverse direction with the GRIN lens and central fiber-bundle cores. Total probe diameter can be as small as 1 mm.

In some embodiments the apparatus further comprises a first optical radiation source connected to or imaged to a proximal end of the light conduit. In some embodiments the light conduit, the photo detector array or image conduit, and the distal end of the probe are configured to back illuminate the target region of a sample in contact or near contact with the distal end of the probe with a light from the first light source to provide a first oblique back illumination of the target region of the sample, and to detect a first phase contrast image from light originating from the first light source and back illuminating the target region of the sample. In some embodiments of the apparatus, the probe comprises a photo detector array.

In some embodiments of the apparatus, the probe comprises an image conduit and a proximal end of the image conduit is connected to or imaged to a photo detector array.

In some embodiments of the apparatus the probe further comprises a second light conduit, wherein a distal end of the second light conduit extends through and ends at the distal end of the probe. In some embodiments a second optical radiation source connected to or imaged to a proximal end of the second light conduit. In some embodiments the second light conduit, the photo detector or image conduit, and the distal end of the probe are configured to illuminate the target region of a sample in contact or ear contact with the distal end of the probe with a light from the second light source to provide a second oblique back illumination of the target region of the sample, and to detect a second phase contrast image from light originating from the second light source and back illuminating the target region of the sample.

In some embodiments of the apparatus, the axis of illumination of the sample with the first light source and the axis of detection of light originating from the first light source and back illuminating the target region of the sample are different. In some embodiments of the apparatus, the axis of illumination of the sample with the second light source and the axis of detection of light originating from the second light source and back illuminating the target region of the sample are different.

In some embodiments of the apparatus, the axis of detection of light originating from the first light source and back illuminating the target region of the sample and the axis of detection of light originating from the second light source and back illuminating the target region of the sample are different. In some embodiments of the apparatus, the axis of detection of light originating from the first light source and back illuminating the target region of the sample and the axis of detection of light originating from the second light source and back illuminating the target region of the sample are the same. In some embodiments of the apparatus, the wavelength of the light from the first light source and the wavelength of light from the second light source are different. In some embodiments the apparatus is configured to detect the first and second images during first and second non-overlapping time intervals. In some embodiments the apparatus is configured to detect the first and second images during first and second overlapping time intervals. In some embodiments the apparatus is configured for illumination of the sample by the first and second light sources with light of different distinguishable wavelengths. In some embodiments the apparatus is configured for illumination of the sample by the first and second light sources with orthogonally polarized light.

In some embodiments of the apparatus the first and second light sources are capable of providing illumination at a wavelength of from 0.2 to 300 μm, from 0.2 to 1 μm, from 0.4 to 0.7 μm, from 0.2 to 0.3 μm, from 0.3 to 0.4 μm, from 0.4 to 0.5 μm, from 0.5 to 0.6 μm, or from 0.6 to 0.7 μm. In some embodiments of the apparatus the first and second light sources are capable of providing illumination at a wavelength that comprises a range selected from at least one of from 0.2 to 300 μm, from 0.2 to 1 μm, from 0.4 to 0.7 μm, from 0.2 to 0.3 μm, from 0.3 to 0.4 μm, from 0.4 to 0.5 μm, from 0.5 to 0.6 μm, or from 0.6 to 0.7 μm. In some embodiments of the apparatus, the light source is selected from a light-emitting diode (LED), a laser, or a superluminescent diode (SLED). In some embodiments of the method, the detecting is by a photo detector array. In some embodiments of the method, the photo detector array is a charge coupled device (CCD) or a CMOS (complementary metal oxide semiconductor) camera sensor. In some embodiments the method comprises using an optical conduit to communicate light in at least one direction selected from toward the sample and away from the sample.

In some embodiments of the apparatus the light source is selected from a light-emitting diode (LED), a laser, or a superluminescent diode (SLED). In some embodiments the apparatus comprises a photo detector array. In some embodiments the photo detector array is a charge coupled device (CCD) or a CMOS (complementary metal oxide semiconductor) camera sensor.

In some embodiments of the apparatus, the apparatus comprises an optical conduit to communicate light in at least one direction selected from toward the sample and away from the sample.

In some embodiments of the apparatus, the axis of illumination of the sample with the first light source and the axis of detection of light originating from the first light source are displaced by from about 0.2 mm to about 3 mm, from about 0.5 mm to about 2.5 mm, from about 1 mm to about 2 mm, from about 1.5 mm to about 2.5 mm, or from about 2 mm to about 3 mm. In some embodiments the axis of illumination of the sample with the first light source and the axis of detection of light originating from the first light source are displaced by about 0.2 mm, about 0.3 mm, about 0.4 mm, about 0.5 mm, about 1.0 mm, about 1.5 mm, about 1.75 mm, about 2.0 mm, about 2.25 mm, about 2.5 mm, about 3.0 mm, about 3.5 mm, about 4.0 mm, or about 5.0 mm. In some embodiments of the apparatus, the axis of illumination of the sample with the second light source and the axis of detection of light originating from the second light source are displaced by from about 0.2 mm to about 3 mm, from about 0.5 mm to about 2.5 mm, from about 1 mm to about 2 mm, from about 1.5 nun to about 2.5 mm, or from about 2 mm to about 3 mm. In some embodiments the axis of illumination of the sample with the second light source and the axis of detection of light originating from the second light source are displaced by about 0.2 mm, about 0.3 mm, about 0.4 mm, about 0.5 mm, about 1.0 mm, about 1.5 mm, about 1.75 mm, about 2.0 mm, about 2.25 mm, about 2.5 mm, about 3.0 mm, about 3.5 mm, about 4.0 mm, or about 5.0 mm. In some embodiments the displacement of the axis of illumination of the sample with the second light source and the axis of detection of light originating from the second light source, and the displacement of the axis of illumination of the sample with the first light source and the axis of detection of light originating from the first light source are the same. In some embodiments the displacement of the axis of illumination of the sample with the second light source and the axis of detection of light originating from the second light source, and the displacement of the axis of illumination of the sample with the first light source and the axis of detection of light originating from the first light source are different.

In some embodiments of the apparatus, the apparatus is configured to allow for imaging a sample in which the object plane of the target region is from the sample surface to about 350 μm below the surface of the sample, from about 100 to about 300 μm below the surface of the sample, from about 150 to about 250 μm below the surface of the sample, from about 175 to about 225 μm below the surface of the sample. In some embodiments it is below the sample surface, greater than about 5 μm below the surface of the sample, greater than about 10 μm below the surface of the sample, greater than about 15 μm below the surface of the sample, greater than about 20 μm below the surface of the sample, greater than about 25 μm below the surface of the sample, greater than about 30 μm below the surface of the sample, greater than about 35 μm below the surface of the sample, greater than about 40 μm below the surface of the sample, greater than about 45 μm below the surface of the sample, greater than about 50 μm below the surface of the sample, greater than about 75 μm below the surface of the sample, greater than about 100 μm below the surface of the sample, greater than about 150 μm below the surface of the sample, greater than about 200 μm below the surface of the sample, greater than about 250 μm below the surface of the sample, greater than about 300 μm below the surface of the sample, or greater than about 350 μm below the surface of the sample.

In some embodiments of the apparatus, the apparatus is configured to create images in which the lateral resolution of the image is from about 0.3 μm to about 2 μm, the lateral resolution of the image is from about 1 μm to about 3 μm, the lateral resolution of the image is from about 2 μm to about 3 μm, the lateral resolution of the image is from about 2 μm to about 5 μm, the lateral resolution of the image is from about 2 μm to about 10 μm. In some embodiments of the apparatus, the lateral resolution of the image is at least about 9 μm, the lateral resolution of the image is at least about 8 μm, the lateral resolution of the image is at least about 7 μm, the lateral resolution of the image is at least about 6 μm, the lateral resolution of the image is at least about 5 μm, the lateral resolution of the image is at least about 4 μm, the lateral resolution of the image is at least about 3 μm, the lateral resolution of the image is at least about 2 μm, the lateral resolution of the image is at least about 1 μm, the lateral resolution of the image is at least about 0.9 μm, the lateral resolution of the image is at least about 0.8 μm, the lateral resolution of the image is at least about 0.7 μm, the lateral resolution of the image is at least about 0.6 μm, the lateral resolution of the image is at least about 0.5 μm, the lateral resolution of the image is at least about 0.4 μm, or the lateral resolution of the image is at least about 0.3 μm.

Also provided is an endoscope, comprising an apparatus according to this disclosure. In some embodiments the endoscope is portable.

Also provided is a system, comprising an apparatus according to this disclosure and a processor for processing images obtained from the apparatus.

Method examples described herein can be machine-implemented or computer-implemented at least in part. Some examples can include a tangible computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

### EXAMPLES

### Example 1

### Monte Carlo Analysis of OBM

As described herein, OBM uses an off axis light source. Illumination light that is multiply scattered in the object is re-directed toward the sample surface and is detected. In this manner, the object plane (defined as the plane that is in focus with respect to the detection optics), is back-illuminated. Because the illumination source is off-axis, the back-illumination flux at the object plane is directed, on average, not quite vertically but with a slight tilt away from the illumination source. That is, the back-illumination is oblique. The illumination source is mimicked by an effective virtual source a distance l*ₛ directly below it (l*ₛ being the transport scattering length of the medium) (FIG. 2). The overlap of the illumination and detection spread functions of the illuminating and detected light is referred to as a “photon banana”. Understanding the distribution of photons in this system will help characterize the geometrical constraints of OBM. In particular, it will help define how far off axis can and/or should the light source be located.

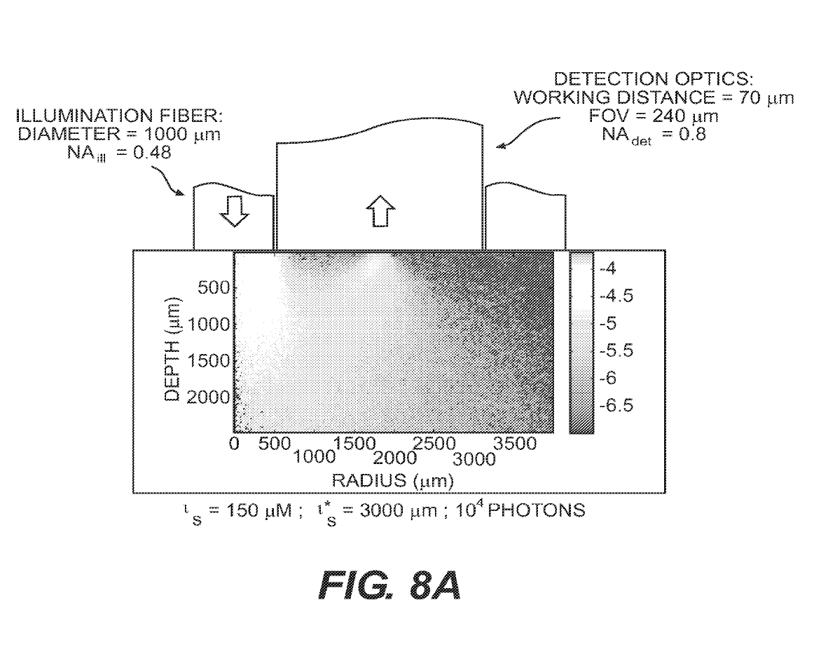

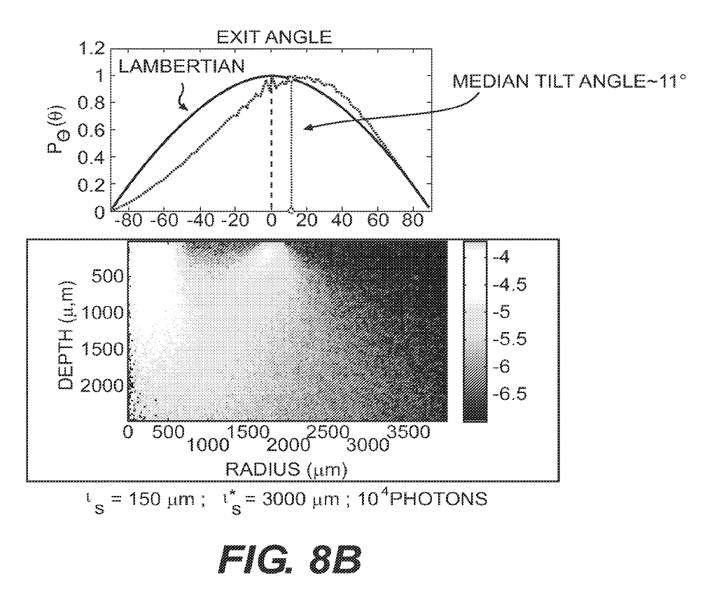

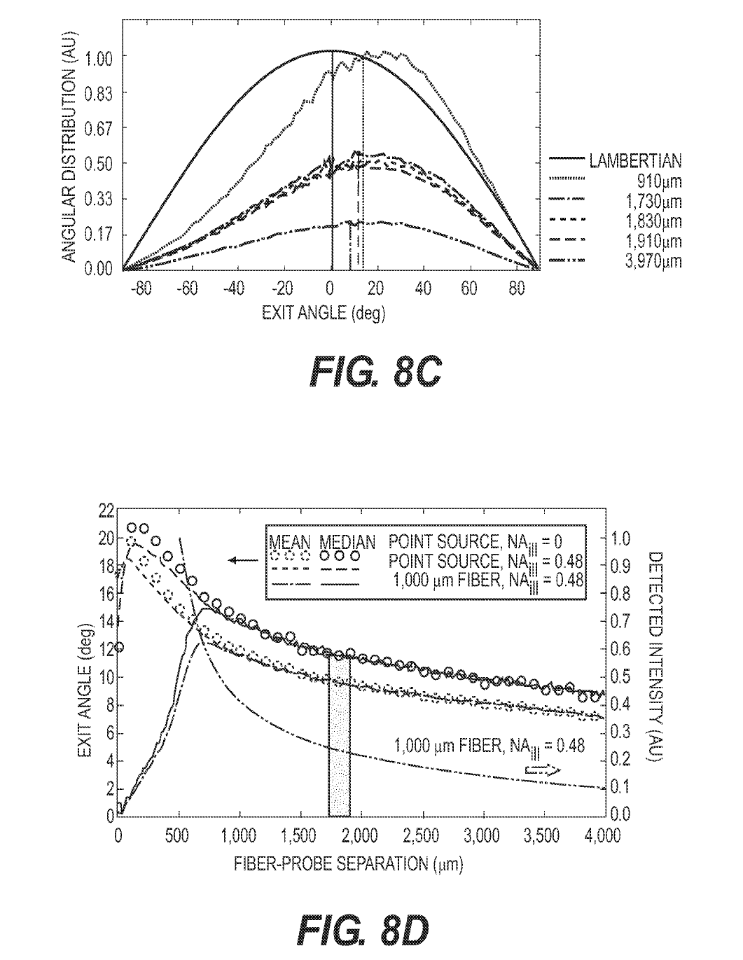

Standard Monte Carlo simulations of light propagation in scattering tissue were performed to validate our claim that light launched into scattering tissue via an off-axis light conduit (e.g., optical fiber) undergoes multiple scattering that leads to an average tilt in the back illumination of an on-axis target region. Only a single source is shown, for simplicity. For our simulations, we chose system parameters that mimicked our OBM endomicroscope device, such as the illumination fiber diameter and numerical aperture (NAᵢₗₗ) the detection field of view (FOV) and numerical aperture (NA_{det}). FIGS. 8a and 8b depict the photon banana, corresponding to the density distribution of photons that are both launched into the tissue by the optical fiber and detected by the endomicroscope distal optics. We also chose scattering parameters lₛ and lₛ* that mimicked biological tissue such as brain tissue. The number of photons in our simulation was chosen to obtain reasonable statistical accuracy in our plots. FIG. 8b depicts the distribution of illumination tilt angles emanating from the target region in the sample. As expected, this distribution is skewed and tilted away from the illumination source. The median tilt angle for our parameters is about 11 degrees.

To further characterize the method, Monte Carlo simulations were used to estimate photon exit angle distributions at different fiber-detector separations. The exit angle corresponds to the tilt angle of the detected photon's path relative to the micro-objective optical axis (positive angles point away from the source). Five fiber-probe separations were considered: 1,830, 1,730 and 1,910 μm correspond to the middle, left, and right extremes of the 2.5×micro-objective FOV, respectively, while 910 and 3,970 μm correspond to roughly half and twice these distances, respectively. The results are shown in FIG. 8(c). The median exit angle for each distribution is noted with a dashed line in FIG. 8(c). A Lambertian exit angle distribution, as would be obtained from isotropic diffuse light in the sample, is also shown for comparison.

Monte Carlo simulations were also used to estimate the dependence of mean and median exit angles and detected intensity as a function of fiber-probe separation. The exit angle corresponds to the tilt angle of the detected photon's path relative to the micro-objective optical axis. The detected intensity is integrated over all exit angles. Three illumination conditions were simulated: a point-source with zero illumination NA, a point source with 0.48 NA, and a 1,000 μm diameter illumination fiber with 0.48 NA, as shown in FIG. 8d. The shaded band represents the actual fiber-probe separation range used in our experiment with the 2.5×micro-objective (FOV=240 μm).

The main conclusion from the Monte Carlo simulations is that, for the parameters used in our experiments, including the light source separation, the back-illumination at the target region of interest is indeed expected to be oblique.

### Example 2

### OBM Images of Onion Skin

To demonstrate the resolution and image quality of the OBM technique, images of onion skin were acquired with a flexible endomicroscope configuration. (FIG. 3.) The endomicroscope setup is based on the use of a flexible fiber-bundle probe containing 30,000 fibers. One probe variant provides a 600 μm field-of-view with 6.5 μm resolution. Another probe variant provides a 240 μm field of view with 2.5 μm resolution. The outer diameter of both probes is 2.8 mm. Imaging depth is 70 μm. Illumination is provided by LEDs, and there are no moving parts. The frame rate of the system is 17 Hz.

Panels (a) and (b) of FIG. 3 are raw images acquired with left and right illumination. Panel (c) is an absorption image (a+b). Panel (d) is a phase gradient image (a- b). Note that panel (d) contains negative values, meaning its zero level is gray. Note that the difference and sum images are very different despite having been obtained simultaneously with the same raw data, demonstrating that phase and absorption images can indeed highlight different sample structures. Note also the DIC-like appearance of the phase gradient image, and the absence of speckle compared to OCT images.

### Example 3

### Images of rodent Colon

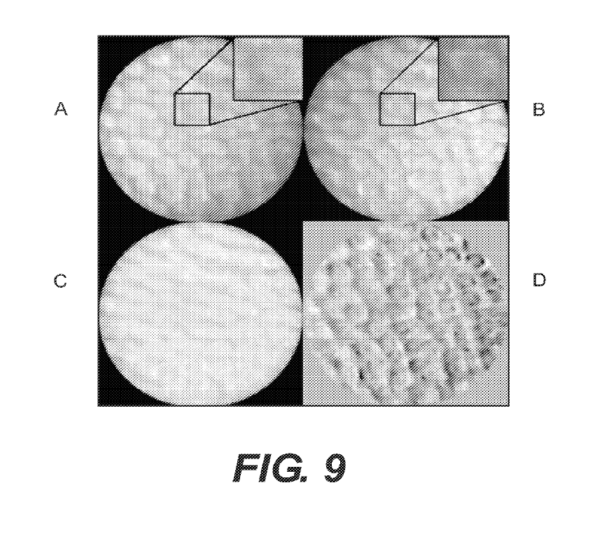

To further demonstrate the resolution and image quality of the OBM technique, as well as to demonstrate its clinical relevance, images of the exposed surface of an excised and fixed rat colon were obtained using the endomicroscope setup described in Example 2. FIGS. 9a and 9b were obtained with left and right illumination, respectively, after core removal (raw core artifacts shown in insets). It is difficult to make out structure without core removal. FIGS. 9c and 9d are the resulting sum (absorption) and difference (phase gradient) images. Both images highlight manifestly different information. In particular, colonic crypts are clearly apparent.

### Example 4

### Images of rodent Intestine

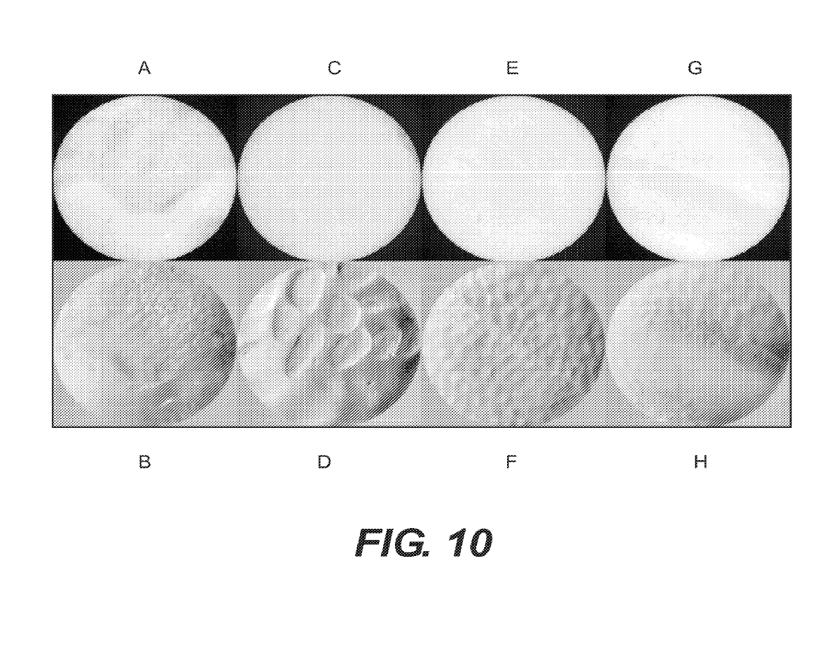

To further demonstrate the resolution and image quality of the OBM technique, as well as to demonstrate its clinical relevance, images of the exposed surface of other regions of the excised and fixed rat intestine were obtained. (FIG. 10.) FIG. 10 shows OBM images of various regions of the rat intestine, acquired with 530 nm LEDs. The top row presents sum images and the bottom row difference (phase gradient) images. Panels (a)-(d) show small intestine (note high resolution content in panel (b); panel (d) highlights epithelial villi). Panels (e)-(h) show large intestine (panel (f) highlights a crease in tissue; panel (h) presumably highlights a blood vessel). Note much higher contrast of phase gradient images compared to relative featurelessness of absorption images. The phase gradient images in these cases are much more revealing than the absorption images. These are high resolution images of thick, unlabeled tissue obtained through a flexible endomicroscope.

### Example 5

### OBM Images Acquired With Hopkins Rod-Lens

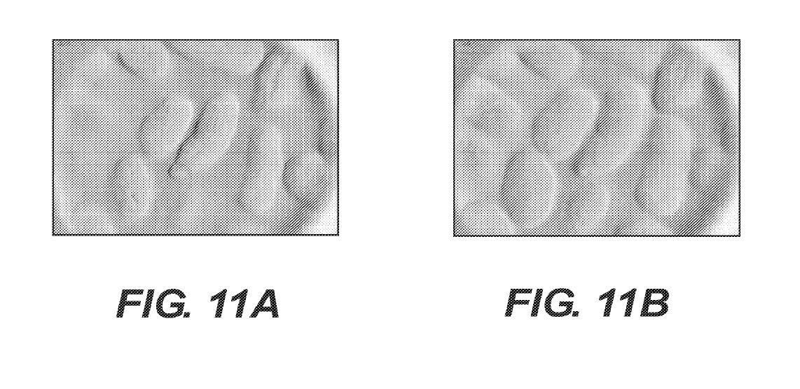

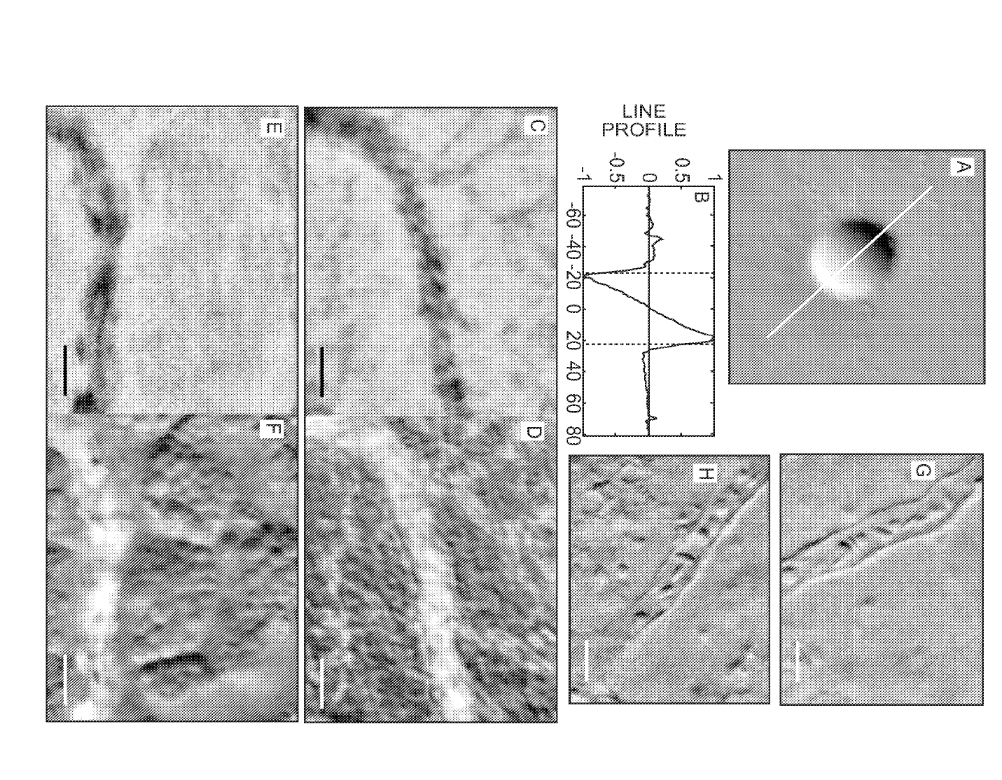

To further demonstrate OBM, phase-gradient images of the same small intestine sample used in Example 3 (FIG. 10), were acquired with a contact mode Hopkins rod-lens using a two-shot implementation. The field of view was 500 μm and the working distance 50 μm. Two frames from a movie taken while focus depth was being adjusted are shown in FIG. 11. For these images green LED illumination was delivered through two diametrically opposed 1 mm fibers. Note the high resolution of the small intestinal villi. This indicates that OBM will be useful for optical biopsy applications.

### Example 6

### Bead and Resolution

To quantify the resolution of OBM, a 45 μm bead was embedded in agarose made scattering by 2 μm beads. As shown in FIG. 12a, not only are the margins of the 45 μm bead crisp and clear but the 2 μm beads embedded in the agarose are themselves very clearly visible.

FIG. 12b is the line profile of the intensity difference image of the bead shown in FIG. 12a. As expected, this line profile reveals the phase gradient induced by the bead.

### Example 7

### Moving Tissues in Chick Embryo

To characterize the ability of the OBM technique to image moving tissues the inventors collected images of blood flow in chick embryos. FIG. 12 shows simultaneous (c) amplitude and (d) phase-gradient images of blood flow in chick embryo (movement in (c) is highlighted in (d)); scale bar=75 μm. Simultaneous (e) amplitude and (f) phase-gradient images acquired with higher magnification fiber bundle (scale bar=30 μm) are also shown. When blood flow is slowed down, the OBM technique provides high resolution images of individual blood cells as shown in FIG. 12 (g) and (h). Note the different types of blood cell morphologies apparent in the images. These images demonstrate the versatility and applicability of the technique.

### Example 8

### Mosaic Images and Movies

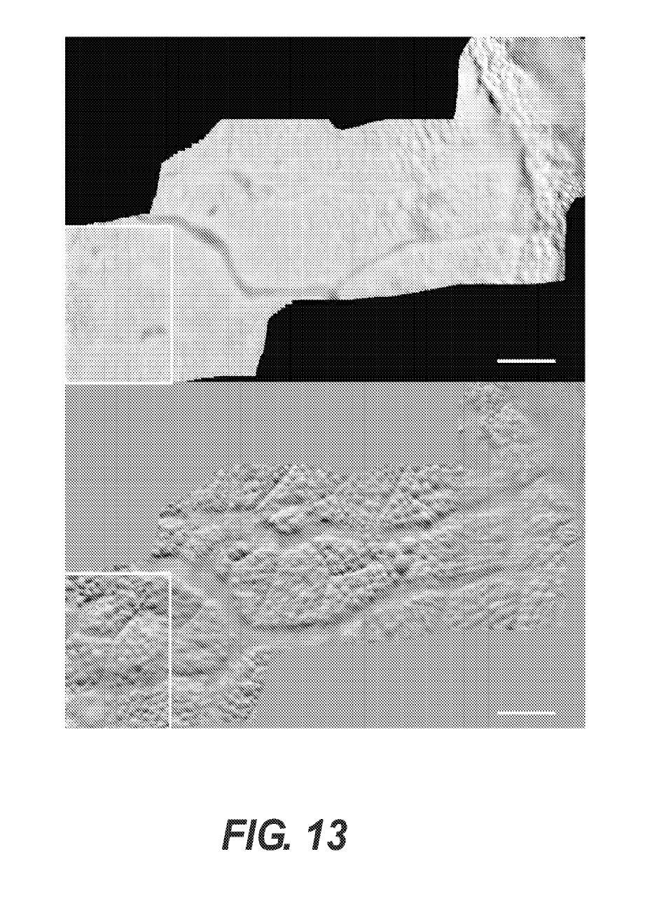

To demonstrate the ability of the OBM technique to acquire images across a large target area, FIG. 13 shows an amplitude (top) and phase-gradient (bottom) mosaic of 300 frames acquired at 17.3 frames per second while manually scanning across a chick embryo. The scale bar is 75 μm and the box indicates the size of a single frame (186 x 139 μm). This example further demonstrates the versatility of the system.

The inventors have also successfully used the OBM technique to create movies. In a first implementation, a phase-gradient movie of subsurface capillaries in a chick embryo (day 11 post-fertilization) was taken using an OBM endomicroscope based on a flexible fiber bundle placed on the embryo surface. The focal depth was varied during acquisition of the movie to demonstrate the pseudo optical-sectioning capacity of instrument. The frame rate was 17.5 Hz (actual frame rate of movie) and the fiber probe was manually scanned over the sample.

In a second implementation, a simultaneous amplitude and phase-gradient movie of vascular and extravascular structure obtained with an OBM endomicroscope probe placed along the yolk membrane in a chick embryo (day 11 post fertilization) was obtained. Again, the frame rate was 17.5 Hz and the fiber probe was manually scanned over the sample.

In a third implementation, a phase-gradient movie of capillaries draining into a venule in a chick embryo (day 7 post fertilization) was obtained. The magnification was 2.5x lower than in videos 1 and 2. The frame rate was 17.5 Hz. The movie was stabilized a posteriori to correct for heart-beat motion. Inter-frame variations (i.e. movement) in the amplitude movie were used to highlight venule and capillaries in the phase-gradient movie.

### Example 9

### Diagnostics

Hyperplasia is a non-neoplastic proliferation of colonic mucosa that results from reduced exfoliation of normal epithelium, and adenoma is a pre-malignant condition that arises from unregulated epithelial growth. These lesions are commonly found on routine screening colonoscopy. Accordingly, the ability to distinguish normal colonic mucosa from that exhibiting hyperplasia or adenomatous changes is useful. Visible morphological structures used to evaluate mucosa during colonoscopy screening include (l) crypt lumens, (c) epithelial cells, (ap) apical border of epithelial cells, (bl) basolateral border of epithelial cells, and (lp) lamina propia.

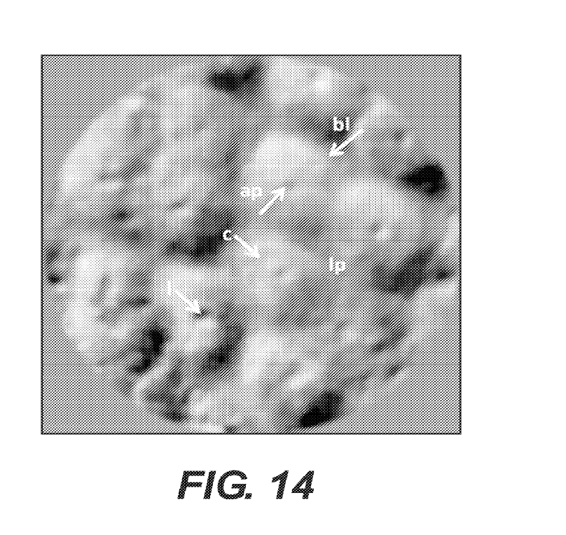

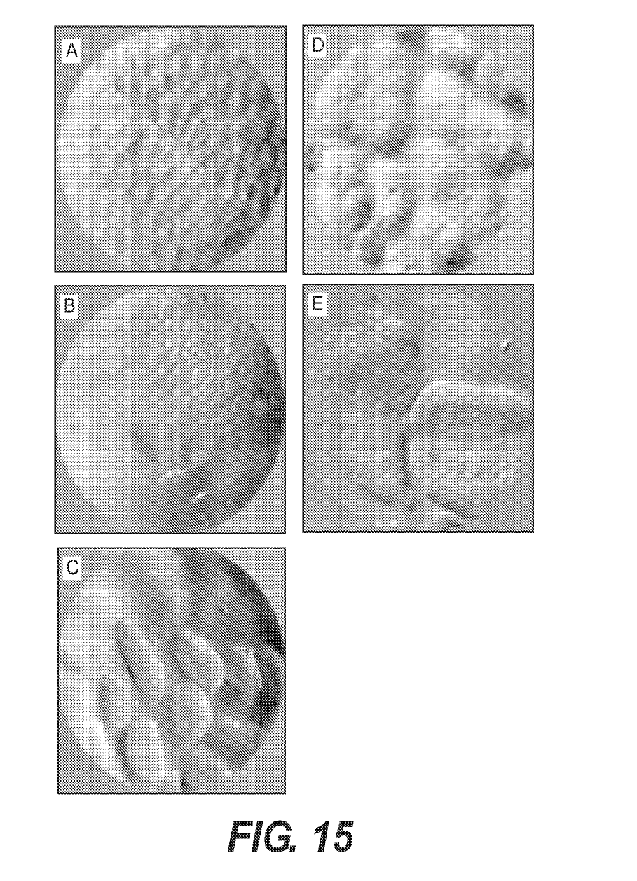

FIG. 14 shows a labeled version of FIG. 15d. Visible morphological structures include (l) crypt lumens, (c) epithelial cells, (ap) apical border of epithelial cells, (bl) basolateral border of epithelial cells, and (lp) lamina propia. (Wang TD et al. “Functional imaging of colonic mucosa with a fibered confocal microscope for real time in vivo pathology” Clin Gastroenterol Hepatol 5(11):1300-1305 (2007).) As shown in FIG. 14, the high resolution phase contrast images generated by OBM allow identification of each of these morphological structures.

### Example 10

### Comparison of Added Versus Subtracted Raw OBM Images

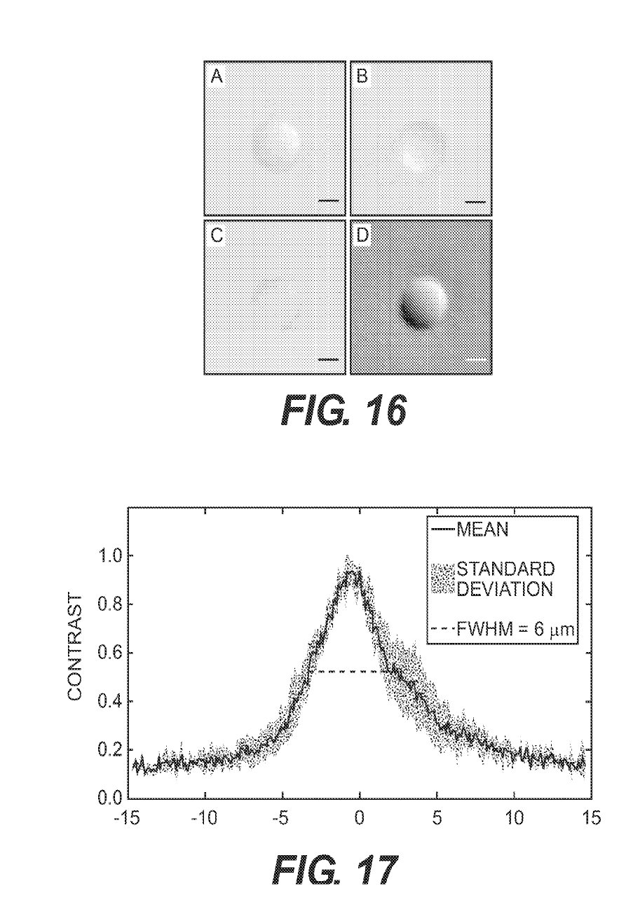

In this example, a 45 μn polystyrene bead embedded in scattering tissue phantoms was analyzed to compare added versus subtracted raw OBM images. Raw images under oblique back-illumination from two opposing directions are shown in FIGS. 16(a) and 16(b). Addition of (a) and (b) cancels phase gradient contrast and emphasizes absorption, as demonstrated in FIG. 16(c). Subtraction of (a) and (b) cancels absorption contrast and emphasizes phase gradients, as shown in FIG. 16(d). The 2 μm beads used to render the tissue phantom scattering are readily visible only when in focus. Lateral resolution is ˜2.6 μm, limited by the fiber core spacing of the imaging fiber bundle (here 3.3 μm, demagnified 2.5× due to the distal micro-objective). Scale bars 20 μm.

### Example 11

### Axial Response

In this example the apparent axial resolution of OBM was explored. A z-stack of OBM phase gradient images was acquired by axially translating a scattering tissue phantom with a step size ˜100 nm. The contrast of five 5.5×5.5 μm (12×12 pixel) regions each bounding single 2 μm diameter beads was computed as (max−min)/(max+min) for every frame in the z-stack. The resulting contrast profiles were normalized and co-registered and are presented in FIG. 17. Increasing z corresponds to deeper imaging.

### Example 12

### Imaging Mouse Lung and Liver Tissue

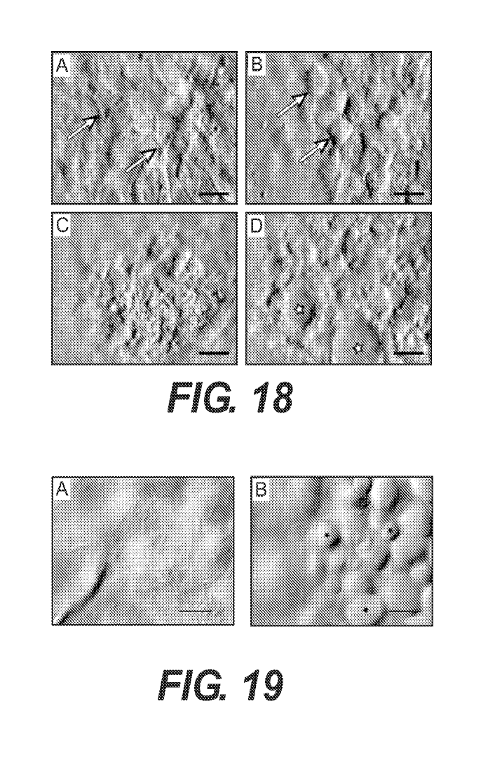

Mouse lung and liver images were acquired using the OBM setup based on a fiber bundle. Fixed mouse liver imaged under phase gradient contrast OBM reveals collagen strands (FIG. 18(aa, arrows) and groups of hepatocytes (FIG. 18(b), arrows). Fixed mouse lung cross-section imaged under phase gradient contrast OBM reveals fine microstructure (FIG. 18(c)) and alveoli (FIG. 18(d), stars). Scale bars are 30 μm. These images demonstrate the usefulness of the OBM technique for analyzing tissue morphology.

### Example 13

### Imaging Rat Flank Skin

OBM was implemented in a bench top microscope to access longer working distances. FIG. 19 shows rat flank skin under phase gradient OBM 50 μm and 100 μm below the surface. FIG. 19(a) is an en face view 50 μm below the surface showing wavy collagen strands. FIG. 19(b) is an en face view 100 μm below the surface showing a cluster of adipocytes (stars). It is noteworthy that contrast remains high even when imaging through ˜30 μm of collagen fibers. Scale bars are 50 μm. These images demonstrate the usefulness of the OBM technique for analyzing tissue morphology at depths of 50 μm and 100 μm below the tissue surface and further demonstrate the general usefulness of the technique in such contexts.

### Example 14

### OBM Depth Penetration in Mouse Brain Slices

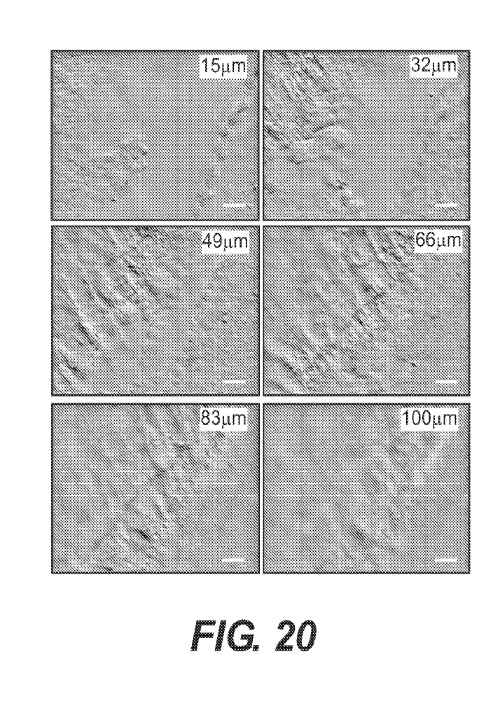

To illustrate OBM penetration depth, the micro-objective and flexible fiber bundle were replaced with a traditional microscope objective (Olympus 40x water immersion, 0.80 NA, working distance 3.3 mm). The two illuminating fibers were guided along the objective housing and placed in contact with the sample. The separation between fiber and objective axes was 4.3 mm. As shown in FIG. 20, a z-stack of a fixed mouse brain slice (sagittal cut through the brain mid-line) illustrates imaging to a depth of 100 μm, beyond which OBM contrast became too weak to reveal structure. In addition to providing greater working distance, the microscope setup afforded improved spatial resolution (˜700 nm, camera pixel limited) compared to the flexible fiber bundle system. Scale bar 30 μm.

### Example 15

### OBM Depth Penetration in Mouse Ventral Skin

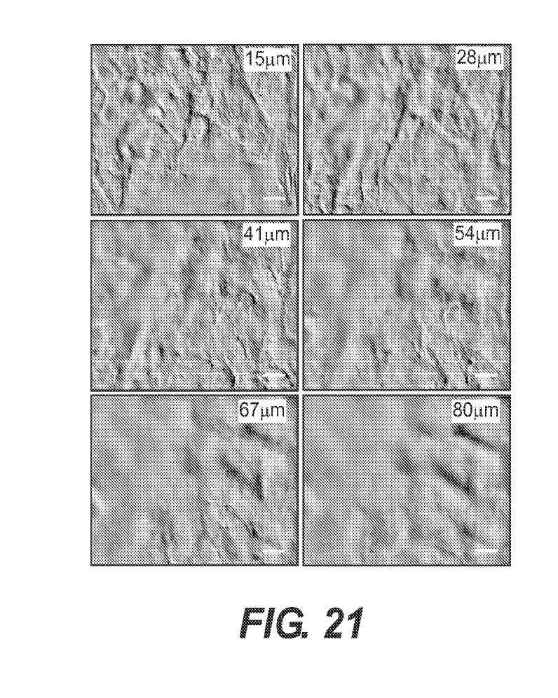

To illustrate penetration depth, OBM was configured in a microscope setup as described in Example 14. As shown in FIG. 21, a z-stack of fixed mouse ventral skin shows keratin filaments in the stratum corneum down to 80 μm, where the deeper layer of stratum granulosum becomes apparent. Scale bar 30 μm.

### Example 16

### Comparison of OBM With Epi-Illumination Reflection Contrast

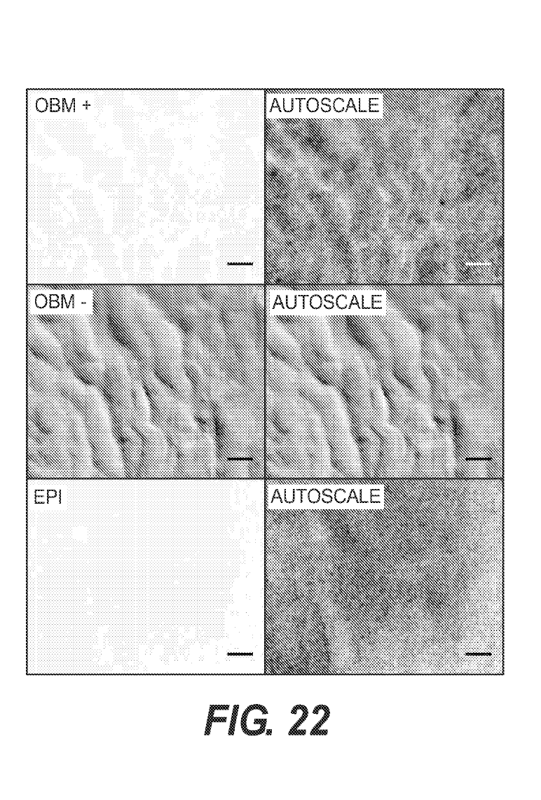

To further characterize OBM, the technique was compared to traditional epi-illumination reflection contrast. To generate epi-illumination reflection contrast, illumination was delivered to the sample directly through the fiber bundle rather than through separate illumination fibers. To minimize extraneous back reflections from the fiber bundle surface, the illumination and detection paths were cross-polarized. The sample was fixed mouse cardiac muscle tissue. As shown in FIG. 22, the phase gradient image (OBM −) exhibits significantly higher contrast than either the absorption image (OBM +) or reflection image (epi). The left panels are displayed with a linear grayscale mapping such that 0 is represented by the middle gray level and the image is scaled to fill the dynamic range of the display. The right panels have been autoscaled by subtracting the minimum values before scaling up to fill the dynamic range of the display, bringing the contrast of the images to 100%. Note that while the removal of the large biases in the absorption and reflection images improves contrast, signal-to-noise ratio remains too low to reveal meaningful structure. Scale bar 20 μm.

### Example 17

### Dual-Camera Multi-Wavelength OBM Endomicroscopy

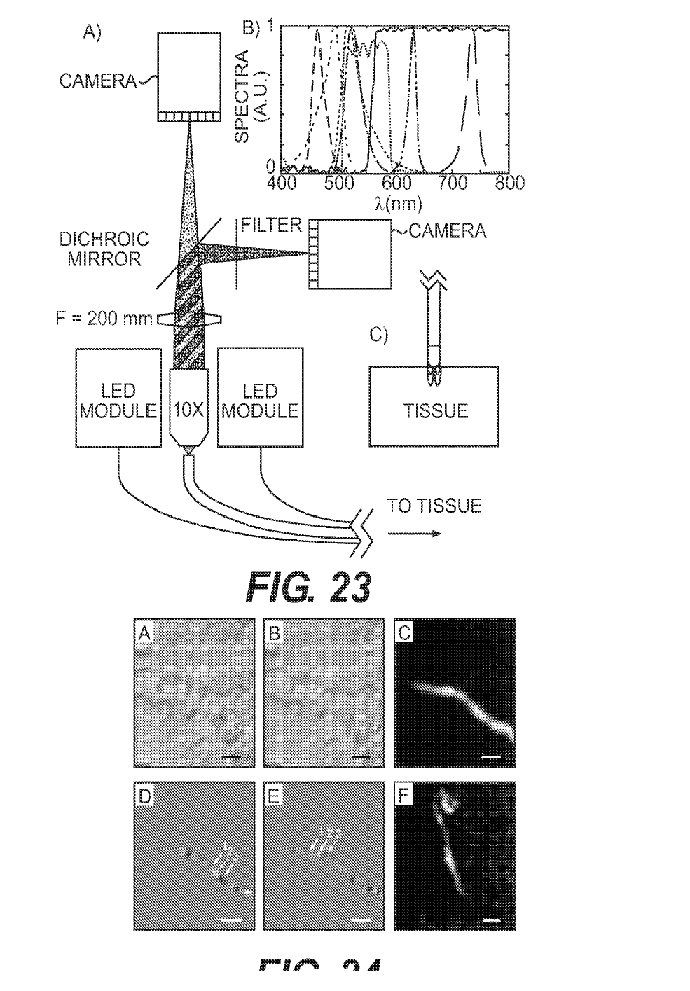

A dual-camera, multi-wavelength setup was used to simultaneously acquire data under different illumination wavelength ranges (see FIG. 23) Two multi-wavelength fiber-coupled light emitting diode (LED) modules (Mightex WFC-H4, SLC-AA04-US) coupled light into optical fibers (Thorlabs BFL48-1000; 0.48 NA; 1000 μm core; 2 m length) through a standard SMA connection. A summary of the available wavelengths and optical powers is shown in Table 1.

TABLE 1
Multi-wavelength illumination parameters

| | Peak | Bandwidth | Optical |
|---|---|---|---|
| Channel | Wavelength (nm) | (1/e², nm) | Power* (mW) |
| blue | 464 | 43 | 22 |
| green | 527 | 60 | 10 |
| red | 632 | 32 | 10 |
| NIR | 736 | 47 | 7.5 |

*after transmission through 2 m optical fiber

The optical fibers were placed in contact with the sample alongside a contact-mode micro-objective (Mauna-Kea Technologies; 2.6 mm diameter; 2.5× magnification; 60 μm working distance; water-immersion; 0.8 NA) coupled to an imaging fiber bundle (30,000 cores; 600 μm active area). The source-detector separation was approximately 1.8 mm. The image at the proximal face of the fiber bundle was relayed to matching monochrome complementary metal oxide semiconductor (CMOS) cameras (PhotonFocus MV1-D1312-160-CL-12, 12-bit mode) with an achromatic objective (Olympus UPLFLN10X2; U Plan Fluorite; 10×, 0.3 NA) and tube lens (Thorlabs AC254-200-A-ML). An image-splitting dichroic beamsplitter (Semrock FF560-FDi01-25×35) was used to send blue and green light to the first camera and red and near infrared (NIR) light to the second. Images were streamed from the camera along a camera-link interface and captured with a dual-base frame grabber (BitFlow KBN-PCE-CL2-D). Frame rate differed by experiment and was typically limited by available illumination power and acceptable signal to noise ratio (SNR).

The dual-camera multi-wavelength OBM endomicroscope setup is illustrated in FIG. 23. As shown in FIG. 23(a) light from two fiber-coupled multi-wavelength LED modules is transmitted along multimode fibers alongside an imaging fiber bundle probe. The image on the proximal face of the fiber bundle is relayed to two high speed CMOS cameras through an image-splitting dichroic beamsplitter such that each camera is sensitive to complementary portions of the visible and NIR spectrum. As shown in FIG. 23(b) normalized emission spectra of four wavelengths was available in each LED module (solid blue, green, red and magenta lines) along with normalized absorption (blue dashed line) and emission (green dashed line) spectra of fluorescein, and transmission spectra of the dichroic mirror (solid black line) and fluorescein emission filter (solid orange line). FIG. 23(c) shows a contact-mode 2.5 x water-immersion micro-objective is fixed to the end of the imaging fiber bundle to give additional magnification and 60 μm working distance. Offset optical fibers deliver light which obliquely back-illuminates the sample at the focal plane. A pre-processing procedure was used to remove the appearance of the fiber bundle cores before

OBM-specific processing was performed. This was done using a segmentation-interpolation algorithm wherein dark regions between the fiber cores were “filled in” with interpolated values based on closest neighbor fiber core signals.

Several modes of operation are available to the dual-camera multi-wavelength setup. Non-limiting examples follow.

### Example 18

### Single Wavelength OBM

One mode of operation is to perform OBM with a single illumination wavelength by synchronously toggling power between the left and right optical fibers. Raw camera frames can then be combined pair-wise to produce either a phase-gradient contrast or amplitude contrast composite image (by subtracting or adding normalized images, respectively). Multiple wavelengths are available to be used individually or in concert; the LED controller allows independent configuration and triggering of each wavelength. This mode utilizes only one of the two cameras, and was used to visualize capillary blood flow through the human eyelid epidermis in vivo (see FIG. 24). Specifically, capillary blood flow was measured in vivo through human eyelid epidermis. FIGS. 24(a) and (b) show individual phase gradient contrast OBM images under simultaneous red and NIR illumination. FIG. 24(c) shows a capillary visualized by a sliding 3-frame temporal variance filter. Multiplying frames in FIG. 24(a, and b) by capillary mask FIG. 24(c) yields the images in FIGS. 24(d, and e), where individual red blood cells are easily distinguished (white arrows). FIG. 24(f) shows another capillary with a more tortuous path extracted from a separate segment from the same video as FIGS. 24(a-e). Scale bars are 20 μm.

### Example 19

### Simultaneous Co-Registered Multi-Wavelength OBM

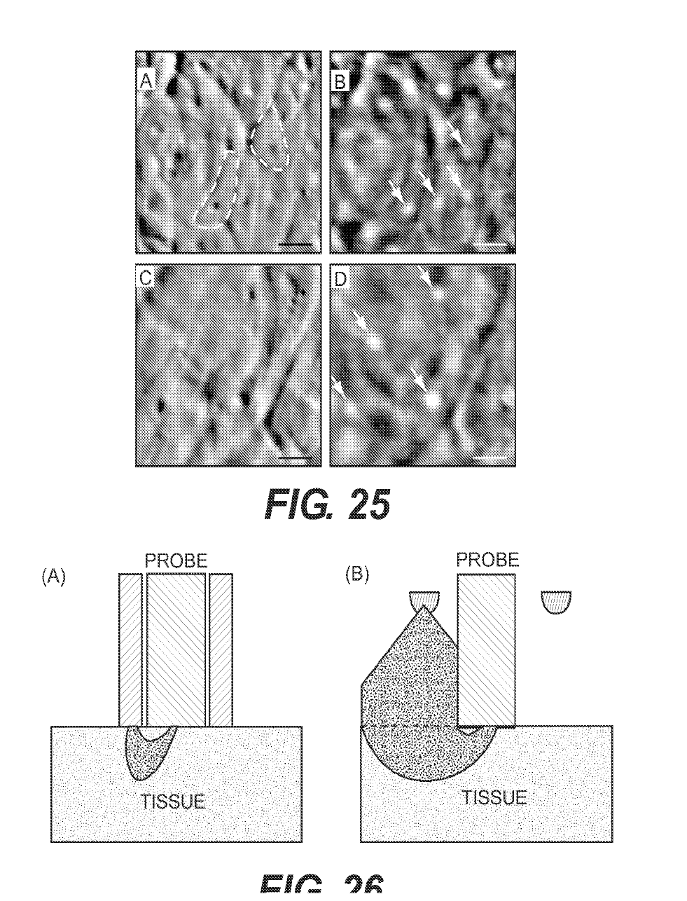

Another available mode of operation to perform OBM is a dual-camera configuration. Such a configuration can be used to simultaneously acquire co-registered OBM images using different wavelengths. In this case four raw images are acquired in the time span of two exposures. This mode was used to simultaneously visualize phase gradient contrast in the red/NIR spectrum and amplitude contrast in the blue/green spectrum. Stratified squamous epithelium of the buccal mucosa was imaged in vivo under the flexible OBM endomicroscope probe. Phase gradient (FIGS. 25(a and c)) images and corresponding amplitude images (FIGS. 25(b and d)) were obtained under red and blue/green illumination, respectively. Scale bars are 30 μm for FIGS. 25(a-b) and 20 μm for FIGS. 25(c-d). The phase gradient images highlight buccal cell borders (dashed lines in FIG. 25(a)), cell nuclei as well as sub-cellular features. The amplitude images reveal cell nuclei in high contrast (arrows in FIGS. 25(b,d)). All images are still frames from a video acquired at 30 fpps (60 fps).

### Example 20

### Recessed Illumination

FIG. 26 is a schematics of different OBM illumination configurations. FIG. 26(a) illustrates llumination delivered via fiber optic conduits in contact with the tissue surface. FIG. 26(b) illustrates illumination delivered by light sources not in contact with the tissue surface. In both cases, illumination is depicted only from one source (left); however, alternative configurations with two or more light sources may also be used. As shown in the figure, OBM can be achieved with light sources that are not necessarily in contact with the sample, but can be recessed by several centimeters. For example, the illumination could be delivered by light sources or fibers that are offset from the optical axis but are set back from the sample surface. In this case, the light sources can diverge over large angles. Illumination obliquity is then assured by the shadow cast by the probe housing itself (as illustrated in FIG. 26(b)). Alternatively, if detection is performed through an objective, the illumination can be delivered through the same objective but producing offset illumination spots at the sample surface that are outside the field of view of the detection optics (as illustrated in Fig XXXa). Alternatively, if detection is performed through a fiber bundle, the illumination can be delivered through the peripheral fiber cores in the bundle and detection can be performed through the central cores of the bundle where imaging is performed via micro-objective (such as a gradient index (GRIN) lens of diameter smaller than the fiber bundle (as illustrated in FIG XXXb).

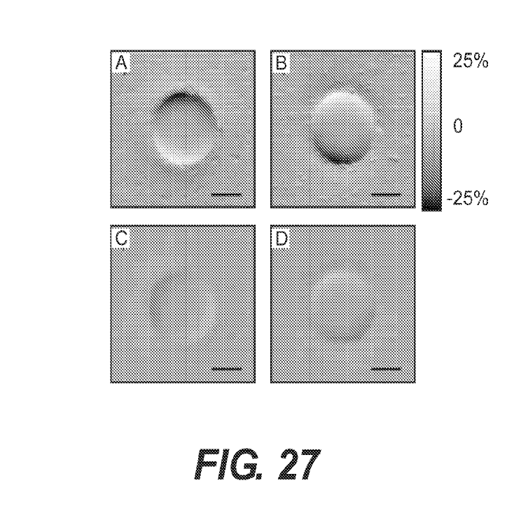

A comparison of OBM with fiber-mediated illumination in contact with the tissue versus non-fiber-mediated illumination not in contact with the tissue is shown in FIG. 27. For this example the sample was a 45 um bead just under the surface of a tissue phantom (comprising 2 um beads in 2% agarose gel, lₛ* ˜1 mm). Images were acquired with a rigid laparascope setup. All images have been phase-gradient OBM processed after averaging approximately 20 frames to reduce shot noise. The camera was a Qlmaging Retiga 2000R. Panels (a) and (b) are OBM images acquired with fiber-delivered LED light (˜650 nm). In (b) the subtraction of images has opposite sign as in (a), leading to a change in sign in the phase gradient. Panels (c) and (d) are OBM images acquired with a 6-element LED flashlight held approximately 2 inches from the sample surface. The 45 um bead is observed but with lower normalized contrast than the fiber illumination. Background 2 um beads remain barely discernible. Scale bar is 20 um. This example demonstrates the usefulness of OBM with a recessed light source and demonstrates that the light source need not be structurally associated with the probe or the detection optics.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### REFERENCES

1. B. Young, W. Stewart, and G. O'Dowd, Wheater's Basic Pathology: A Text, Atlas and Review of Histopathology, 5th ed. Churchill Livingstone, 2009, p. 348.

2. J. Van Dam, “Novel methods of enhanced endoscopic imaging,” British Medical Journal, vol. 52, no. 4, 2003.

3. T. D. Wang and J. Van Dam, “Optical Biopsy: A New Frontier in Endoscopic Detection and Diagnosis,” Clin. Gastroenterol. Hepatol., vol. 2, no. 9, pp. 744-753, 2004.

4. F. Zernike, “How I discovered phase contrast,” Science, vol. 121, pp. 345-349, 1955.

5. G. Nomarski, “Microinterferometre differentuiel a ondes polarisees,” J. Phys. Radium., vol. 16, p. S9, 1955.

6. R. D. Allen, G. B. David, and G. Nomarski, “The Zeiss-Nomarski differential interference equipment for transmitted light microscopy,” Z. Wiss. Mikrosk., vol. 69, pp. 193-221, 1969.

7. J. G. Dodd, “Interferometry with Schlieren microscopy,” Appl. Opt., vol. 16, pp. 470-472, 1977.

8. R. Hoffman and L. Gross, “Modulation contrast microscopy,” Appl. Opt., vol. 14, pp. 1169-1176, 1975.

9. R. Yi, K. K. Chu, and J. Mertz, “Graded-field microscopy with white light,” Opt. Express, vol. 14, pp. 5191-5200, 2006.

10. K. K. Chu, R. Yi, and J. Mertz, “Graded-field autoconfocal microscopy,” Optics Express, vol. 15, no. 5, pp. 2476-2489, 2007.

11. S. B. Mehta and C. J. R. Sheppard, “Quantitative phase-gradient imaging at high resolution with asymmetric illumination-based differential phase contrast.,” Optics letters, vol. 34, no. 13, pp. 1924-6, Jul. 2009.

12. S. S. Kou, L. Waller, G. Barbastathis, and C. J. R. Sheppard, “Transport-of-intensity approach to differential interference contrast (TI-DIC) microscopy for quantitative phase imaging.,” Optics letters, vol. 35, no. 3, pp. 447-9, Feb. 2010.

13. M. R. Arnison, K. G. Larkin, C. J. R. Sheppard, N. I. Smith, and C. J.

Cogswell, “Linear phase imaging using differential interference contrast microscopy,” Journal of Microscopy, vol. 214, no. 1, pp. 7-12, 2004.

14. G. Popescu, R. R. Dasari, and M. S. Feld, “Hilbert phase microscopy for investigating fast dynamics of transparent systems,” Optics Letters, vol. 30, no. 10, pp. 1165-1167, 2005.

15. G. Popescu et al., “Fourier phase microscopy for investigation of biological structures and dynamics,” Opt. Lett., vol. 29, pp. 2503-2505, 2004.

16. H. Ding and G. Popescu, “Instantaneous Spatial Light Interference Microscopy.,” Optics express, vol. 18, no. 2, pp. 1569-75, Jan. 2010.

17. J. Pawley, Ed., Handbook of Biological Confocal Microscopy, 3rd ed. Springer, 2006, p. 988.

18. J. Schmitt, “Optical coherence tomography (OCT): a review,” IEEE Journal of selected topics in quantum electronics, vol. 5, no. 4, pp. 1205-1215, 1999.

19. A. Dubois, L. Vabre, A. C. Boccara, and E. Beaurepaire, “High resolution full-field optical coherence tomography with a Linnik microscope,” Appl. Opt., vol. 41, pp. 805-812, 2002.

20. A. Latrive and A. C. Boccara, “In vivo and in situ cellular imaging full-field optical coherence tomography with a rigid endoscopic probe,” Biomedical optics expressOptics Express, vol. 2, no. 10, pp. 2897-2904, 2011.

21. D. Kang, D. Yelin, B. E. Bouma, and G. J. Tearney, “Spectrally-encoded color imaging,” Opt. Express, vol. 17, no. 17, pp. 15239-15247, 2009.

22. J. G. Fujimoto, “Optical coherence tomography for ultrahigh resolution in vivo imaging,” Nature Biotechnology, vol. 21, no. 11, pp. 1361-1367, 2003.

23. M. Xu and L. V. Wang, “Photoacoustic imaging in biomedicine,” Review of Scientific Instruments, vol. 77, no. 4, p. 041101, 2006.

24. B. R. Masters and P. So, Eds., Handbook of Biomedical Nonlinear Optical Microscopy. Oxford University Press, USA; 1 edition, 2008, p. 896.

25. S. Hu and L. V. Wang, “Photoacoustic imaging and characterization of the microvasculature,” Journal of Biomedical Optics, vol. 15, no. May, p. 011101, 2010.

26. K. Koenig, A. Ehlers, I. Riemann, S. Schenkl, R. Buckle, and M. Kaatz, “Clinical Two-Photon Microendoscopy,” Microscopy Research and Technique, vol. 402, no. 2006, pp. 398-402, 2007.

27. J. C. Jung, A. D. Mehta, E. Aksay, R. Stepnoski, M. J, and M. J. Schnitzer, “In Vivo Mammalian Brain Imaging Using One- and Two-Photon Fluorescence Microendoscopy,” Journal of Neurophysiology, pp. 3121-3133, 2008.

28. Y. Wu, J. Xi, Y. Chen, M. J. Cobb, M.-jun Li, and X. Li, “Fiber-optic Endomicroscopy for Intrinsic Two-photon Fluorescence Imaging of Epithelial Cells and Tissues,” 2009 CONFERENCE ON LASERS AND ELECTROOPTICS AND QUANTUM ELECTRONICS AND LASER SCIENCE CONFERENCE CLEOQELS 2009 VOLS 15, vol. 1, pp. 30-31, 2009.

29. P. Kim, M. Puoris'haag, D. Coté, C. P. Lin, and S. H. Yun, “In vivo confocal and multiphoton microendoscopy,” J. Biomed. Opt., vol. 13, no. 1, p. 010501, 2008.

30. D. A. Boas et al., “Imaging the body with diffuse optical tomography,” IEEE signal processing magazine, no. November, pp. 57-75, 2001.

31. E. M. C. Hillman and S. a Burgess, “Sub-millimeter resolution 3D optical imaging of living tissue using laminar optical tomography.,” Laser & photonics reviews, vol. 3, no. 1-2, pp. 159-179, February 2009.

32. W. Groner et al., “Orthogonal polarization spectral imaging: a new method for study of the microcirculation.,” Nature medicine, vol. 5, no. 10, pp. 1209-12, Oct. 1999.

33. J. Lindert, J. Werner, M. Redlin, H. Kuppe, H. Habazetti, and A. R. Pries, “OPS Imaging of Human Microcirculation : A Short Technical Report,” J. Vasc. Res., vol. 39, pp. 368-372, 2002.

34. S. Santos et al., “Optically sectioned fluorescence endomicroscopy with imaging through a flexible fiber bundle,” Journal of Biomedical Optics, vol. 14, no. June, pp. 1-3, 2009.

35. T. N. Ford, D. Lim, and J. Mertz, “Fast optically sectioned fluorescence HiLo endomicroscopy,” J. Biomed. Opt., vol. 17, no. 2, p. 021101-1-021101-7, 2012.

36. R. Yi, K. K. Chu, and J. Mertz, “Graded-field microscopy with white light,” Opt. Express, vol. 14, no. 12, pp. 5191-5200, 2006.

37. J. C. Mertz, Introduction to Optical Microscopy. Roberts and Company Publishers, 2009, p. 413.

38. L. V. Wang and H.-i Wu, Biomedical Optics: Principles and Imaging, 1st ed. Wiley-Interscience, 2007, p. 376.

39. E. Beaurepaire and J. Mertz, “Epifluorescence collection in two-photon microscopy,” Applied optics, vol. 41, no. 25, pp. 5376-5382, 2002.

40. V. Tuchin, Tissue Optics: Light scattering methods and instruments for medical diagnosis, 2nd ed. SPIE Publications, 2007, p. 882.

41. M. Oheim, E. Beaurepaire, E. Chaigneau, J. Mertz, and S. Charpak, “Two-photon microscopy in brain tissue: parameters influencing the imaging depth.,” Journal of neuroscience methods, vol. 111, no. 1, pp. 29-37, October 2001.

42. C. Schwartz and A. Dogariu, “Backscattered polarization patterns determined by conservation of angular momentum.,” Journal of the Optical Society of America. A, Optics, image science, and vision, vol. 25, no. 2, pp. 431-6, Feb. 2008.

43. V. Rossetto, “General framework for multiple scattering of polarized waves including anisotropies and Berry phase,” Physical Review E, vol. 80, no. 5, pp. 1-15, November 2009.

44. F. Jaillon and H. Saint-Jalmes, “Scattering coefficient determination in turbid media with backscattered polarized light.,” Journal of biomedical optics, vol. 10, no. 3, p. 034016, 2005.

45. M. J. Raković et al., “Light backscattering polarization patterns from turbid media: theory and experiment.,” Applied optics, vol. 38, no. 15, pp. 3399-408, May 1999.

46. N. Bozinovic, C. Ventalon, T. Ford, and J. Mertz, “Fluorescence endomicroscopy with structured illumination,” Optics Express, vol. 16, no. 11, pp. 4603-4610, 2008.

47. A. M. De Grand et al., “Tissue-like phantoms for near-infrared fluorescence imaging system assessment and the training of surgeons,” Journal of biomedical optics, vol. 11, no. February, p. 014007, 2006.

1. A method of creating a phase contrast image, comprising:
illuminating the target region of a sample with a first light source to provide a first oblique back illumination of the target region of the sample, and
detecting a first phase contrast image from light originating from the first light source and back illuminating the target region of the sample.

2. The method of claim 1, wherein light from the first light source is the only light illuminating the sample when the first phase contrast image is detected from light originating from the first light source and back illuminating the target region of the sample.

3. The method of claim 2, further comprising illuminating the sample with a second light source to provide a second oblique back illumination of the target region of the sample, and
detecting a second phase contrast image from light originating from the second light source and back illuminating the target region of the sample.

4. The method of claim 3, further comprising creating a difference image of the target region of the sample by subtracting the second phase contrast image of the target region of the sample from the first phase contrast image of the target region of the sample.

5. The method of claim 3 or claim 4, further comprising creating an absorption contrast image of the target region of the sample by adding the first phase contrast image of the target region of the sample to the second phase contrast image of the target region of the sample.

6. The method of any one of claims 1-5, wherein the axis of illumination of the sample with the first light source and the axis of detection of light originating from the first light source and back illuminating the target region are different.

7. The method of any one of claims 1 and 3-6, wherein the axis of illumination of the sample with the second light source and the axis of detection of light originating from the second light source and back illuminating the target region are different.

8. The method of any one of claims 3-7, wherein the axis of detection of light originating from the first light source and back illuminating the target region and the axis of detection of light originating from the second light source and back illuminating the target region are different.

9. The method of any one of claims 3-7, wherein the axis of detection of light originating from the first light source and back illuminating the target region and the axis of detection of light originating from the second light source and back illuminating the target region are the same.

10. The method of any one of claims 3-9, wherein the wavelength of the light from the first light source and the wavelength of light from the second light source are different.

11. The method of any one of claims 1-10, wherein the wavelength of the light from the first and second light sources is from 0.2 to 300 μm.

12. The method of any one of claims 1-11, wherein the light source(s) is selected from a light-emitting diode (LED), a laser, a supercontinuum light source, or a superluminescent diode (SLED).

13. The method of any one of claims 1-12, wherein the detecting is by a photo detector array.

14. The method of claim 13, wherein the photo detector array is a charge coupled device (CCD) or a CMOS (complementary metal oxide semiconductor) camera sensor.

15. The method of any one of claims 1-14, comprising using an optical conduit to communicate light in at least one direction selected from toward the sample and away from the sample.

16. The method of any of claims 1-15, wherein the optical conduit to communicate light in at least one direction selected from toward the sample and away from the sample is selected from a fiber, an arrangement of fibers, a fiber bundle, a rigid lens, an arrangement of rigid lenses, a gradient index (GRIN) lens, or an arrangement of GRIN lenses.

17. The method of any of claims 1-16, wherein the same optical conduit communicates light toward the sample and away from the sample.

18. The method of any of claims 1-17, wherein different components of the same optical conduit communicates light toward the sample and away from the sample.

19. The method of any one of claims 1-18, wherein the axis of illumination of the sample with the first light source and the axis of detection of light originating from the first light source are displaced by from about 0.2 mm to about 10 mm.

20. The method of any one of claims 3-19, wherein the axis of illumination of the sample with the second light source and the axis of detection of light originating from the second light source are displaced by from about 0.2 mm to about 10 mm.

21. The method of any one of claims 1-20, wherein the object plane of the target region is from the surface to about 300 μm below the surface of the sample.

22. The method of any one of claims 1-21, wherein the lateral resolution of the image is from about 0.1 μm to about 10 μm.

23. The method of any one of claims 3-22, comprising detecting the first and second images during first and second non-overlapping time intervals.

24. The method of any one of claims 3-22, comprising detecting the first and second images during first and second overlapping time intervals.

25. The method of claim 24, wherein the first and second light sources illuminate the sample with light of different distinguishable wavelengths.

26. The method of claim 25, wherein the images of different distinguishable wavelengths are separated by a wavelength separator and directed onto separate camera sensors.

27. The method of claim 25, wherein the images of different distinguishable wavelengths are separated by a wavelength separator and directed onto different portions of a same camera sensor.

28. The method of claim 24, wherein the first and second light sources illuminate the sample with orthogonally polarized light.

29. The method of claim 28, wherein the images of orthogonal polarization are separated by a polarization separator and directed onto separate camera sensors.

30. The method of claim 28, wherein the images of orthogonal polarization are separated by a polarization separator and directed onto different portions of a same camera sensor.

31. The method of any one of claims 4-30, wherein the difference image is axially resolved.

32. The method of any one of claims 1-31, further comprising obtaining a series of two or more images and combining the images to provide a composite image larger than the field of view a single image.

33. The method of any one of claims 1-32, comprising creating a phase contrast image of gastrointestinal tissue and examining the tissue to assess at least one of the presence and the absence of indicators of a disease.

34. The method of claim 33, wherein the gastrointestinal tissue is colonic mucosa disease is at least one of hyperplasia and adenomatous changes.

35. The method of any one of claims 1-32, comprising creating a phase contrast image of lung tissue and examining the tissue to assess at least one of the presence and the absence of at least one indicator of a disease.

36. The method of any one of claims 1-32, comprising creating a phase contrast image of liver tissue and examining the tissue to assess at least one of the presence and the absence of at least one indicator of a disease.

37. The method of any one of claims 1-32, comprising creating a phase contrast image of bladder tissue and examining the tissue to assess at least one of the presence and the absence of at least one indicator of a disease.

38. The method of any one of claims 1-32, comprising creating a phase contrast image of skin tissue and examining the tissue to assess at least one of the presence and the absence of at least one indicator of a disease.

39. The method of any one of claims 1-32, comprising creating a phase contrast image of brain tissue and examining the tissue to assess tissue morphology.

40. The method of any one of claims 1-32, comprising creating a phase contrast video of blood flow to assess blood flow velocity.

41. The method of any one of claims 1-32, comprising creating a phase contrast video of blood flow to assess the cell count of at least one blood cell type.

42. An apparatus for creating a phase contrast image of a sample, comprising:
a probe comprising 1) an optical radiation source or a first light conduit, and 2) a photo detector array or image conduit, and 3) a distal end,;
wherein the light conduit, the photo detector array or image conduit, and the distal end of the probe are configured to back illuminate the target region of a sample in contact or near contact with the distal end of the probe with a light from the first light source to provide a first oblique back illumination of the target region of the sample, and to detect a first phase contrast image from light originating from the first light source and back illuminating the target region of the sample.

43. The apparatus of claim 42, wherein the distal end of the optical radiation source or first light conduit extend to the distal end of the probe.

44. The apparatus of claim 42, wherein the distal end of the optical radiation source or first light conduit is recessed from the distal end of the probe by up to 10 cm.

45. The apparatus of any one of claims 42-44, wherein the distal end of the photo detector array or image conduit is recessed from the distal end of the probe.

46. The apparatus of claim 42, wherein the probe comprises a first light conduit and the apparatus further comprises a first optical radiation source connected to or projected to a proximal end of the first light conduit.

47. The apparatus of claim 42, wherein the probe comprises a photo detector array.

48. The apparatus of claim 42, wherein the probe comprises an image conduit and a proximal end of the image conduit is connected to or imaged to a photo detector array.

49. The apparatus of any one of claims 42-48, wherein the probe further comprises:
a second optical radiation source or a second light conduit;
wherein the second optical radiation source or second light conduit, the photo detector array or image conduit, and the distal end of the probe are configured to illuminate the target region of a sample in contact or near contact with the distal end of the probe with a light from the second light source to provide a second oblique back illumination of the target region of the sample, and to detect a second phase contrast image from light originating from the second light source and back illuminating the target region of the sample.

50. The apparatus of claim 49, wherein the distal end of the optical radiation source or first light conduit extends to the distal end of the probe.

51. The apparatus of claim 49, wherein the distal end of the optical radiation source or first light conduit is recessed from the distal end of the probe by up to 10 cm.

52. The apparatus of any one of claims 49-51, wherein the distal end of the photo detector array or image conduit is recessed from the distal end of the probe

53. The apparatus of claim 49, wherein the probe comprises a second light conduit and the apparatus further comprises a second optical radiation source connected to or imaged to a proximal end of the first light conduit.

54. The apparatus of any one of claims 42-53, comprising at least three optical radiation sources or a light conduits, wherein the at least three optical illumination sources or light conduits are located at distinct locations around the probe such that each is capable of creating oblique back illumination enabling the measurement and display of phase gradients in different directions relative to the others.

55. The apparatus of any one of claims 42-54, wherein the axis of illumination of the sample with the first light source and the axis of detection of light originating from the first light source and back illuminating the target region of the sample are different.

56. The apparatus of any one of claims 49-55, wherein the axis of illumination of the sample with the second light source and the axis of detection of light originating from the second light source and back illuminating the target region of the sample are different.

57. The apparatus of any one of claims 49-56, wherein the axis of detection of light originating from the first light source and reflected from the sample and the axis of detection of light originating from the second light source and illuminating the target region of the sample are different.

58. The apparatus of any one of claims 49-56, wherein the axis of detection of light originating from the first light source and reflected from the sample and the axis of detection of light originating from the second light source and back illuminating the target region of the sample are the same.

59. The apparatus of any one of claims 49-58, wherein the wavelength of the light from the first light source and the wavelength of light from the second light source are different.

60. The apparatus of any one of claims 49-59, configured to detect the first and second images during first and second non-overlapping time intervals.

61. The apparatus of any one of claims 49-60, configured to detect the first and second images during first and second overlapping time intervals.

62. The apparatus of claim 61, configured for illumination of the sample by the first and second light sources with light of different distinguishable wavelengths.

63. The apparatus of claim 61, configured for illumination of the sample by the first and second light sources with orthogonally polarized light.

64. The apparatus of any one of claims 42-63, wherein the first and second light sources are capable of providing illumination at a range of wavelengths comprising from 0.2 to 300 μm.

65. The apparatus of any one of claims 42-64, wherein the light source is selected from a light-emitting diode (LED), a laser, a supercontinuum light source, or a superluminescent diode (SLED).

66. The apparatus of any one of claims 42-65, comprising a photo detector array.

67. The apparatus of claim 66, wherein the photo detector array is a charge coupled device (CCD) or a CMOS (complementary metal oxide semiconductor) camera sensor.

68. The apparatus of any one of claims 1-67, comprising an optical conduit to communicate light in at least one direction selected from toward the sample and away from the sample.

69. The apparatus of any one of claims 42-68, configured so that the axis of illumination of the sample with the first light source and the axis of detection of light originating from the first light source are displaceable by from about 0.2 mm to about 5 mm.

70. The apparatus of any one of claims 49-69, configured so that the axis of illumination of the sample with the second light source and the axis of detection of light originating from the second light source are displaceable by from about 0.2 mm to about 5 mm.

71. The apparatus of any one of claims 42-70, configured to obtain images of object planes of the target region from the surface of the sample to about 300 μm below the surface of the sample.

72. The apparatus of any one of claims 42-71, that creates images laterally resolved at from about 0.3 μm to about 10 μm.

73. The apparatus of any one of claims 42-72, wherein at least one of 1) the distal end of the first optical radiation source or first light conduit, and 2) the distal end of the second optical radiation source or second light conduit, and 3) the distal end of the photo detector array or image conduit, extend through and end at the distal end of the probe.

74. The apparatus of any one of claims 42-72, wherein at least one of 1) the distal end of the first optical radiation source or first light conduit, and 2) the distal end of the second optical radiation source or second light conduit, and 3) the distal end of the photo detector array or image conduit, is recessed from the distal end of the probe by up to 5 cm.

75. An endoscope comprising an apparatus according to any one of claims 42-74.

76. The endoscope of claim 75, wherein the endoscope is portable.

77. A system comprising an apparatus according to any one of claims 42-74 or an endoscope according to claim 75 or claim 76, and a processor for processing images obtained from the apparatus.

78. A method of creating at least one of a phase contrast image of a target region of a sample and a difference image of two phase contrast images of a target region of a sample, comprising:
providing a sample comprising a target region; using an apparatus of any one of claims 42-74 to create at least one phase contrast image of the target region of the sample using a method according to any one of claims 1-41, and optionally creating a difference image from the two or more contrast images of the target region of the sample.

79. A phase contrast image created by the method of any one of claims 1-41 and 78.
